(19) United States
(12) Patent Application Publication
(10) Pub. No.: US 2022/0142813 A1
(43) Pub. Date: May 12, 2022

(54) MOBILE ITCHING TREATMENT DEVICE WITH INTERFACE

(71) Applicant: DERMAPHARM AG, Grünwald (DE)

(72) Inventor: Daniel BÜNGER VON WURMB, Mönchengladbach (DE)

(73) Assignee: DERMAPHARM AG, Grünwald (DE)

(21) Appl. No.: 17/419,565

(22) PCT Filed: Jan. 23, 2020
(86) PCT No.: PCT/EP2020/051560

§ 371 Date: Jun. 29, 2021

(30) Foreign Application Priority Data
Jan. 23, 2019 (EP) 19153229.0

(51) Int. Cl. A61F 7/00; G16H 40/67; G16H 20/30

(52) U.S. Cl. CPC A61F 7/007; G16H 40/67; G16H 20/30; A61F 2007/0086; A61F 2007/0052; A61F 2007/0071; A61F 2007/0096; A61F 2007/0078; A61F 2007/0093

(57) ABSTRACT

The invention preferably relates to a device for hyperthermal treatment of itching and/or herpes diseases comprising a control device on which at least two treatment programs are stored and an interface for a selection of one of the at least two treatment programs by means of a mobile device, so that after actuation of an operating element the control device heats a treatment surface according to the selected treatment program to a treatment temperature between 40°-65° C. and holds the treatment temperature for a treatment duration between 1 and 12 seconds.

The invention preferably relates to a device for hyperthermal treatment of itching and/or herpes diseases comprising a control device on which at least two treatment programs are stored and an interface for a selection of one of the at least two treatment programs by means of a mobile device, so that after actuation of an operating element the control device heats a treatment surface according to the selected treatment program to a treatment temperature between 40°-65° C. and holds the treatment temperature for a treatment duration between 1 and 12 seconds.

### Background and State of the Art

Itching (pruritus) is a subjectively unpleasant sensory perception related to the skin or mucous membrane. It can be locally delimited or affect the entire body. Itching is often accompanied by a burning, stinging or tingling sensation, which the person affected often tries to alleviate by scratching, scrubbing, pressing, kneading or rubbing. Therefore itching is often accompanied by other pathological skin phenomena such as scratching, open wounds, crust formation and skin infections. Experts assume that itching is mediated via pain receptors in the skin and is transmitted to the brain via the autonomic nervous system. The causes of itching can be very diverse. In addition to dry skin, lack of moisture or allergies, itching may also arise due to external influences and skin irritations, such mosquitoes bites or contact with nettles. Itching may be a reaction to chemical, mechanical or thermal stimuli. It may be caused by external irritation, such as the effects of chemical substances, e.g. histamine (mosquito bite), apamine (bee sting), by an allergic immune reaction, by pressure or friction, or by heat or sunlight, wheals, urticaria and other skin reactions associated with itching. From a medical point of view, the causes or underlying illnesses that lead to itching cover a broad spectrum of dermatological and internal diseases.

For the medicinal treatment of the symptoms of itching a number of medications or cosmetic products are known. For example, essential oils such as menthol, thymol or camphor are used extensively to provide short-term cooling. In addition, skin care products like creams or lotions may have analgesic effects by increasing the moisture content of the skin. Furthermore, antihistamines are helpful therapeutic options, for example comprising the administration of dimetindene maleate or mepyramine. Additional medication include topical glucocorticoids, anaesthetics, zinc ointments, calcineurin inhibitors or capsaicin. For the treatment of wasps or bee stings, the stinging sites may also be treated with ammonia solution, but this provides only a brief relief of itching and only slightly reduces the swelling.

However, it is also known from the state of the art to reduce the development of itching by applying a quantity of heat to the insect bite. A device for a local, thermal treatment of especially mosquito bites is described in EP 1231875 B1 and WO 01/34074 A1. The device has a heating plate with a size of approx. 0.2 cm², which is brought to a temperature between 50° C. and 65° C. while the heating plate is in contact with the insect bite. This hyperthermic treatment provides lasting relief from itching. On the one hand, the heat application causes the breakdown of thermolabile toxins of the insects, which cause the itching. On the other hand, the heat transfer results in a masking of the itching by other temperature-dependent skin sensations. Thus as a result of such treatments secondary injuries to the skin, for example inflammation of the insect bite due to scratching, may also be effectively avoided. In this way the hyperthermic treatment also effectively reduces the development of wheals accompanying an insect bite.

The possible applications of a hyperthermic treatment also extend to herpes diseases.

Herpes is commonly known as an acute, primary or secondary viral disease of the skin and mucous membranes caused by infection by the herpes virus. It usually occurs as locally grouped vesicles, especially in the face (herpes facialis), on the cheeks (herpes buccalis), on the nose (herpes nasalis) and on the genitals (herpes genitalis). The initial infection often occurs in early childhood and without being detected, but leads to the formation of antibodies and often to masking of the virus and colonization of body tissues, which leads to relapses in virus carriers with a weakened immune system. Lip herpes creams, e.g. with the active ingredient acyclovir, are used to alleviate pain and itching in recurrent herpes labialis. These creams should be applied at the first signs of herpes (burning, itching, tension and redness) as they inhibit the growth of certain viruses (antiviral, anti-viral DNA polymerase inhibitor). The duration of treatment is generally 5 days. The disadvantage of such creams is that side effects and intolerances may occur or the hoped-for effects may not occur in whole or in in part.

From DE 102005002946 A1 a device for a hyperthermic treatment of herpes diseases is known. The device comprises a heating plate with a preferred size of 20 mm², which is heated to 49° C.-53° C. for a treatment duration of preferably 10-15 sec. During the treatment duration the heating plate contacts the affected skin area of the lips, for example the reddened area or the position where blisters have already formed. The heat application leads on the one hand to a containment of the multiplication of the causative pathogens by means of a neutralizing effect on the herpes simplex viruses. On the other hand, the short-term heat treatment leads to masking of the itching of the herpes disease by stimulating temperature-sensitive nerves. The device is thus characterized by a reduction of the symptoms of the herpes disease such as burning, the appearance of swelling, redness or itching.

The devices for hyperthermic treatment known from the prior art are characterized by a wide range of possible uses for the relief of symptoms of insect bites, herpes diseases, jellyfish stings or other diseases associated with itching. However, the devices also have disadvantages.

In exceptional cases, for example, the desired treatment temperature may be exceeded.

In the prior art it is therefore known to monitor the treatment temperature by means of temperature sensors. However, damage to the device, for example due to the ingress of moisture, can impair the control circuit of the monitoring electronics. This is especially the case if the monitoring of the treatment temperature is integrated into the regular control circuit. In this case, it cannot be ruled out that the temperature may rise above the desired treatment temperature. Depending on the contact position of the heating plate or treatment surface, undesirable side effects may occur. Even a short-term temperature increase of more than 65° C. can cause lasting damage to the affected skin areas. This is especially the case for sensitive skin areas, such as lips during a herpes treatment or thinner skin areas in case of insect bites.

From US 2007/0049998 A1, a device for hyperthermic treatment of skin complaints is known, which heats a treatment surface via a temperature-controlled heating element to temperatures of 38° C.-67° C. for a duration of at least 5 seconds, but typically over a longer period of time, and uses a fuse to protect against overheating. This type of protection against overheating has the disadvantage that if the fuse is triggered due to overheating, it has to be replaced. In addition, there is no redundant safety mechanism and if the fuse fails, the treatment surface may overheat for a longer period of time. Furthermore, temperatures above 60° C. or above, especially over a longer period of a few seconds or longer, are perceived as very unpleasant and can lead to skin damage. As a result the treatment success is jeopardized, because treatments are terminated preliminary due to an unpleasant skin sensation caused by the high temperatures, thus endangering the success of the therapy. The device is based on the therapeutic idea of destroying germs and killing irritating agents of the skin by applying heat. However, the treatment times and/or temperatures required for this are not suitable for providing lasting relief of itching through targeted stimulation of certain receptors and the modification of the immune system. Temperatures below 42° C. are on the other hand unsuitable to achieve effects of a therapeutic nature beyond the sensation of heat.

US 2011/0184502 A1 describes a heating pad for various, partly medical applications, which electrically generates temperatures of 38° C.-71° C. for at least several minutes. Non-resetting thermal fuses are proposed in series as a redundant safety feature. Thus a redundant safety mechanism is available, but it is not reversible and must be replaced after triggering the fuse. A second disadvantage of using non-resetting thermal fuses is that they only melt, when a temperature above a threshold value is reached. This means that non-resetting thermal fuses do not react until this critical temperature is reached after a certain reaction time and thus may be too late compared to an (electrical) fuse. An (electrical) fuse already trips at an electric current above a threshold value, which could cause a too high temperature if the current is present longer times. In addition, both the temperature range and the duration of the heating process are certainly relevant for a large number of applications, but are unsuitable for sustainably alleviating pruritus by applying heat.

Moreover, especially in the prior art, no device is known which combines the advantages of modern mobile devices, e.g. high computing power, networking, but also the ability to serve as an energy source for physical application, with a device for the treatment of the above-mentioned itching disorders in a safe and reliable way.

Mobile devices, especially smartphones, are not only used today for their original purpose, e.g. making phone calls. Rather, it is nowadays common practice to combine as many applications as possible in a small portable device. Today's smartphones are also used as cameras, players, readers, computer games, navigation devices and much more.

In the prior art connections of mobile control units for a power supply or for a control of treatment devices are known for some cases.

US 2018/369064 A1 discloses a portable device for improving blood flow by means of vibration, optionally supported by an application of heat or cold. To control the portable device a mobile device can be provided on which an ‘app’ is installed to this end.

From the CN 107 280 850 A a device for the treatment of mosquito bites by means of a heat application is known. The device exhibits an USB interface (e.g. for connection to a mobile phone) for a power supply or for charging a battery. A use of the mobile device to control the treatment device via the USB interface is not disclosed.

KR 101 722 904 B1 discloses a device for a hyperthermal treatment of mosquito bites. By means of an interface, the device can be recharged by mobile devices. Furthermore, a control of the heating unit of the device is disclosed by means of an “app”. Disadvantageously, there is an increased risk of incorrect treatment or damage in case of incorrect execution or manipulation of the app. For medical applications, especially during the treatment of itching or herpes, mobile devices have so far played a minor role.

It would therefore be desirable to be able to provide a device that implements the benefits of hyperthermic treatment for the large number of the above-mentioned diseases while minimizing safety risks.

### Objective of the Invention

An objective of the invention was to eliminate the disadvantages of the prior art. In particular, it was an objective of the invention to provide a device for a hyperthermal treatment of itching or herpes, which can exploit the advantages of mobile devices, e.g. with respect to their user-friendliness, computing power or networkability, while at the same time ensuring high safety standards.

### SUMMARY OF THE INVENTION

The objective of the invention is solved by a device according to the independent claim. The dependent claims relate preferred embodiments of the invention.

In a preferred embodiment, the invention thus relates a device for a hyperthermal treatment of itching and/or herpes comprising
a. at least one treatment surface
b. preferably an operating element and
c. a control device, which is configured, after actuation of the operating element, to regulate the treatment surface in a heating-up phase to a treatment temperature between 40°-65° C. by heating at least one heating element according to predetermined treatment parameters of a treatment program and to maintain said treatment temperature for a treatment duration between 1 and 12 seconds

characterized in that

at least two different treatment programs specifying different treatment parameters are stored on the control device, and wherein the device has at least one interface for connection to a mobile device, and the device is configured for a selection of one of the at least two treatment programs by said mobile device, so that the selected treatment program is executed after actuation of the operating element. Preferably the operating element is on the device itself. However, in some embodiments, the operating element may also be provided by the mobile device. For example, the touch screen of the mobile device can be used as an operating element.

In a further aspect, the invention therefore also relates to a device for the hyperthermal treatment of itching or herpes comprising
a. at least one treatment surface
b. a control device, which is configured to regulate the treatment surface in a heating-up phase to a treatment temperature between 40°-65° C. by heating at least one heating element according to predetermined treatment parameters of a treatment program and to maintain the treatment temperature for a treatment duration between 1 and 12 seconds
   wherein at least two different treatment programs specifying different treatment parameters are stored on the control device and wherein the device has at least one interface for connection to a mobile device and the device is configured for selection of one of the at least two treatment programs by the mobile device, so that the selected treatment program is executed after actuation of an operating element, which is present on the device or the mobile device,.

Research has shown that the application of heat by means such a device can be successfully used to treat itching.

To this end the device according to the invention is preferably applied to the affected skin areas. After contact of the skin area with the treatment surface, a control device ensures that the temperature of the treatment surface is regulated according to the invention. The treatment temperature preferably relates to the temperature which is present in the skin area of the patient. To this end the treatment surface is preferably first heated up to a treatment temperature between 40° C. and 65° C.

It is preferred that the heating-up phase does not require a longer period of time. Preferably, the heating-up phase should not exceed 12 seconds, and especially preferred not more than 3 seconds. In a preferred embodiment of the invention, the heating-up phase is 1 second to 5 seconds, preferably less than 3 seconds and especially 1 to 2 seconds. The desired temperature may be reached particularly fast by such short heating phase. Thus, healing effects can be achieved preferentially without unnecessarily supplying heat to a user and/or increasing the effective time required for a treatment. In addition, the amount of heat applied during the treatment can be controlled with a particular high precision. Due to a targeted and significantly faster heating-up phase than is common with state-of-the-art devices, a particularly high acceptance of the subjects and thus a reliable therapy success can be achieved. Advantageously, it is avoided that the skin areas of the subjects are unnecessarily irritated during a therapeutically ineffective heating-up phase. Instead, the predetermined treatment temperature is reached quickly and reliably.

Following the heating-up phase, the temperature of the treatment surface is maintained at the predetermined treatment temperature for a predetermined treatment period of 1 to 12 seconds.

By regulating the treatment surface at a temperature between 40° C. and 65° C. during a treatment phase, heat is transferred in a well-defined way. Pain and other unpleasant sensations such as tension or itching can be relived to a surprisingly effective extend. The effect may also be exploited in case of insect bites or herpes diseases, where the relief is additionally based on thermal neutralization of the poisons of the insects or herpes-causing viruses.

On the other hand, the heat causes nerve stimulation, which greatly reduces the subjective perception of pain or itching in the affected sites. Surprisingly, the heat transfer results in a masking of the unpleasant sensation by other temperature-dependent skin sensations.

Unlike conventional methods of treatment, the aim is to additionally regulate the pain receptors and to activate the free nerve endings of the C fibers through the preferred heat treatment. The C fibers refer in particular to the slowly conducting nerve fibers of the somatosensory system and are responsible for the sensation of pain. In this process the free ends of the C fibers, which are also known as nociceptors, play an important role. The nerve endings of the fibers are activated by tissue hormones (e.g. histamine, serotonin, substance P). In addition, mast cells in the vicinity of the nerve endings can become involved in the process by releasing the mediator tryptase. In particular, knowledge of the mechanism of action in itching or herpes is exploited to regulate the sensory perception triggered by the fibers in a surprising manner by heat treatment.

Furthermore, it has been recognized that a particularly pronounced masking of the unpleasant sensations can be attained if the thermal and capsaicin receptors TRPV1 and TRPV2 are locally activated simultaneously at the affected skin sites. TRPV1 is involved in acute heat-induced pain in healthy skin and regulates, for example, the sensation of heat at temperatures about 45° to 50° C. In addition, TRPV2 is activated in the case of particularly severe painful heat sensations that occur at temperatures above 52° C. The activation threshold of TRPV1 is between 40° C. and 45° C., whereas that of TRPV2 is between 50° C. and 53° (Yao et al 2011, Somogyi et al 2015, Cohen et al 2014, Mergler et al 2014).

In the sense of the invention, the term “treatment parameters” refers preferably to those parameters or characteristics which characterize the course of temperature regulation of the treatment surface during a treatment. Preferred treatment parameters include the treatment temperature, the treatment duration or the duration of a heating-up phase. However, also other parameters may represent treatment parameters. If the device is equipped with a separate preheating-up phase in order to bring the treatment surface to a preheating temperature below the treatment temperature, the duration of the preheating-up phase or the preheating temperature may represent further treatment parameters. Preferably, the treatment parameters relate to any parameters which may influence the temperature of the treatment surface at a given time.

A “treatment program” preferably refers to an algorithm which, based on defined treatment parameters, regulates the temperature curve of the treatment surface by means of the control device. The treatment program thus preferably comprises a defined combination of treatment parameters as well as, if necessary, additional control algorithms to guarantee the specified treatment parameters. For example, it may be preferable to keep the treatment temperature by means of a temperature sensor in a predefined range using a feedback control. In such a case, the treatment program may preferably include additional instructions for a feedback control of a treatment temperature in addition to the specified treatment temperature for the treatment surface.

In a preferred embodiment, the treatment parameters defined by the treatment programs include the treatment temperature and the treatment duration, whereby a duration for the heating-up phase is preferably additionally defined as treatment parameter. These parameters are particularly relevant to the treatment and define the temperature curve, which is essential for the success of the treatment.

The treatment program can be software-based and/or hardware-based. The device is characterized by the fact that at least two treatment programs are stored on the control device. The at least two treatment programs differ particularly preferably in at least one treatment parameter. For example, a first treatment program can specify a first treatment temperature, while a second treatment program specifies a second treatment temperature. Similarly, the treatment programs can of course also differ in the treatment duration, the duration of the heating-up period or another treatment parameter. The treatment programs may also differ in two or more treatment parameters.

The possibility of selecting different treatment programs allows to optimally apply the device for different treatment purposes. For example, a first treatment program may be optimized for the treatment of lip herpes, while a second treatment program may be especially effective in reducing itching after insect bites.

The different treatment programs may furthermore be used to adapt the treatment to the needs and preferences of different users.

Studies have shown that the individual sensation of heat treatment is perceived differently amongst users. For instance, some subjects perceive a temperature of over 48° C. as painful, while other subjects describe temperatures of 50° C. or more as pleasant and itch-reducing. Therefore, by providing at least two treatment programs, more individual treatments may be carried out and the success of treatments can be increased.

For the selection of the treatment program, the device comprises at least one interface which is configured for the selection of one of at least two treatment programs by a mobile device.

A mobile device is preferably understood to refer to mobile terminals which, due to their size and weight, can be carried with minor physical effort and can therefore be used on the move.

Preferably, these are electronic terminals for mobile, network-independent data, voice and image communication, navigation or the like. Particularly preferred mobile devices are, for example, a laptop, a mobile phone, a smartphone, a tablet computer, a notebook and/or a Smartwatch.

Such mobile devices are very popular and characterized especially by attractive operating options and elevated user-friendliness. The integration of mobile devices for the operation of a hyperthermic treatment device leads to a number of advantages. Firstly, a selection of preferred treatment programs can be made more clearly and easily, and the mobile device can also display previous successful treatment programs to the user. In addition, several user profiles can be stored on the mobile device, each showing the preferred treatment programs for the respective user. The integration of a mobile device is seen to significantly increase the frequency of treatment and thus the success of treatment.

In addition, the possibility of connecting the device to a mobile device makes it possible to create an especially compact device for treating itching and/or herpes. For example, it is not necessary for the device itself to have many operating or display elements. Instead, functions such as the selection of treatment programs, the display of a successful treatment, the temperature curve of the treatment surface or other functions that facilitate the execution and control of the treatment may be taken over by the mobile device. Nor does the device itself need to have high computing power or control capacities. Instead, the control device can essentially be designed to perform safety-relevant functions such as regulating the temperature of the treatment surface. A longer-term storage of data or calculations based on such data can moreover be outsourced to a mobile device.

As a result it is possible to provide a particularly compact, cost-effective and efficient device for the treatment of itching or herpes, which at the same time benefits from the extensive operating, display and computing capacities of modern mobile devices.

According to the invention, the device can be connected via the existing interface.

The presence of an interface, preferably means that the device is configured to interact with and/or be connected to a mobile device.

The interface may relate to a physical location where the device can be connected to another device. It may also be another optional connection of the device to another device to exchange data, signals and/or power. These may preferably include electrical signals or signals that can be converted into electrical signals by the device.

A connection enabled by such an interface preferably comprises three elements: an interface of the mobile device, said interface of the device and a transmission channel between the two. For example, the transmission channel may be a cable-based connection and/or a wireless transmission channel. It may also be preferred that two interfaces are designed in the form of a socket and a matching plug and thus a direct connection can be established, which preferably also enables a mechanically stable connection between the device and a fmobile device. In this case and in case of a cable-based connection possibility, the interface of the device would preferably comprise a socket and/or a plug.

Depending on whether the interface is wireless or wired or is intended for direct plug and socket connection, the interface comprises differently designed elements.

The interface can be a standardized interface, such as Bluetooth, Lightning, USB, WLAN etc. However, it can also be an interface, which is custom developed for the device.

The interface can be wireless or wired. A cable can be suitable for the transmission of electrical charge carriers, and include for example a copper cable, but a cable may equally be used for the transmission of optical signals and include for example an optical fiber (cable). A wireless transmission can preferably be based on the transmission of electromagnetic waves over the entire spectral range, for example light signals can be used for a wireless transmission, but likewise short waves, ultra -short waves and/or decimetric waves.

If the interface is e.g. USB, the interface of the device is preferably a USB socket of the device suitable for a USB connection. Furthermore, it is preferably in this case that suitable data converters, line drivers, power supplies and/or processors are available on the part of the device in order to use the USB socket as intended, in particular to establish a USB connection with a mobile device. In particular, a connection of electrical components of the device with the interface shall be moreover ensured. Said example serves to illustrate an interface of the device and as a principle can be transferred to other types of interfaces of the device.

The interface of the device is preferably configured at least for receiving of data or information allowing the selection of one of at least two treatment programs.

To receive data, the device may comprise a data receiving unit, which may also be integrated in the interface. The data receiving unit can receive data coming from the mobile device via the interface, convert the data, if necessary, into a suitable data format and pass the data on to the control device for further processing and/or a storage medium for storage in a suitable manner. A person skilled in the art knows possibilities to implement suitable units routinely.

The device, or its control device and interface, is preferably configured in such a way that a selection of one of at least two treatment programs can be made by means of a mobile device. The corresponding information of the selection is preferably transmitted from the mobile device via the interface to the control device.

The selection preferably means that the treatment programs are stored on the control device and that the data transmission by means of the mobile device can determine which of the stored treatment programs is executed when the operating element is actuated.

With such a selection, it is in particular not possible for the mobile device to change the stored treatment programs via the interface. This means that neither a change of the treatment parameters nor the compilation of a new treatment program as a combination of stored treatment parameters by a mobile device is intended. Such changes are preferably excluded by appropriate circuitry.

In terms of temperature regulation, the device is thus self-sufficient and independent of the mobile device.

The inventors have recognized that particularly high safety standards can be guaranteed by such a limitation of the regulatory possibilities of a mobile device.

An alternative approach would be to transfer treatment-relevant parameters from the mobile device via the interface to the control device of the device. At first sight more individual treatment programs could be put together to improve the treatment experience. However, the inventors have recognized that a safe performance of hyperthermic treatment may be severely impaired by such a regulatoin.

Mobile devices are characterized by a high degree of networkability and typically have a variety of different software (‘apps’) with different origins and functions. Both circumstances cause mobile devices, as open systems, to be inherently vulnerable to security risks.

It may happen, for example, that after an update of the operating software a software for connection to the device is executed incorrectly. Furthermore, a targeted manipulation by computer viruses or other hacker attacks cannot be excluded.

The provision of external software on a mobile device, which may exclude such scenarios, would involve a high effort and cost. Nevertheless, a residual risk would continue to exist.

Departing from such an approach, a particularly high safety standard is implemented in a simple and cost-efficient manner by limiting the regulatory rights of the mobile device to a selection of predetermined treatment programs, as provided for in the invention.

Since software on the mobile device cannot influence the safety-relevant treatment parameters of the device, the software can meet significantly lower safety standards without affecting the safety of the device for hyperthermic treatment of itching or herpes.

Even in the event of a failure of the operating software on the mobile device or a targeted manipulation attempt, only a selection of the pre-installed treatment programs may be conducted via the interface of the device at any one time.

In a preferred embodiment, the device is thus configured in such a way that the mobile device is excluded from a change of the treatment parameters, which are specified by the treatment programs. To this end various technical solutions are known to a person skilled in the art.

For example, the treatment programs may be predefined in the firmware of the control device in such a way that a change via the interface is generally excluded. In the sense of the invention, the firmware is preferably understood to relate to software, i.e. instructions for a computer-implemented procedure, which is embedded in the control device, which may be preferably a microprocessor. That means the firmware preferably comprises software which is functionally connected with the hardware of the device, i.e. in particular with the at least one heating element and, if applicable, temperature sensors. Preferably the firmware is executed upon booting the device and takes over the control and monitoring function of hardware components of the device.

Therefore, within the firmware preferable only a parameter for the selection of a treatment program may be changed via the interface by an appropriate command, but not the stored treatment programs as such.

As a result, the treatment itself will be always and regardless of a connection with a mobile device carried out according to the treatment parameter ranges specified by the treatment programs.

The treatment temperature preferably corresponds to a constant temperature, which is in the mentioned range between 40° C. and 65° C. Said treatment temperature is preferably kept constant during the treatment phase. Constant preferably shall also include a fluctuation of the temperature within a specified tolerance range, for example of ±10%, ±5%, ±3% or ±1% or less.

The treatment phase preferably refers to the period during which the temperature of the treatment surface exhibits the treatment temperature, which may be in the range of 40° C. to 65° C. Preferably, the treatment phase lasts 1 to 12 sec, particularly preferably the treatment phase lasts between 2 sec and 12 sec, 1 sec and 10 sec, especially preferably between 3 sec and 5 sec. It is especially preferred that the treatment phase designates a continuous period of time. However, it is also possible that the treatment phase is briefly interrupted if the temperature is driven in form of a ramp. In this case, the period of the treatment phase is preferably understood as the time during which the temperature of the treatment surface is in fact in the range of the treatment temperature. After ending the treatment phase, preferably no more heating of the at least one heating element by the control device takes place, so that the temperature of the treatment surface falls below the treatment temperature.

In the sense of the invention, the treatment surface preferably refers to the surface of the device which is heated to the treatment temperature during the treatment and is in direct thermal contact with the skin area. The treatment surface may represent a continuous surface. It may also be preferred that the treatment surface consists of several non-contiguous partial surfaces. The size of the treatment surface preferably depends on the illness and the size of the skin areas affected by the symptoms of the illness comprising an itching sensation.

In the case of insect bites, the size of the treatment surface is preferably between 10 mm² and 100 mm², and particularly preferably between 20 mm² and 60 mm². For a treatment of herpes, the treatment surface is preferably between 10 mm² and 80 mm², especially preferably between 20 mm² and 50 mm². Furthermore, it is particularly preferred that the treatment surface for these smaller areas of skin is circular. The sizes and geometries of the treatment surface chosen in this way allow for a treatment that is optimally adapted to the cause, optimizing therapeutic efficiency and thus contributing to a more sustainable treatment success.

The size of the treatment surface preferably refers to the total contact surface over which a part of the skin experiences a thermal impulse. In case of a treatment surface consisting of several partial areas, the size of the treatment surface preferably corresponds to the sum of the individual partial surfaces. Such a division into partial areas can be advantageous for certain manifestations of herpes or itching, as well as for the treatment of certain body parts.

It is preferable that the treatment surface is brought to the desired treatment temperature with the aid of at least one heating element. In a preferred embodiment, the treatment surface corresponds to the surface of a heating plate which is heated by means of a heating element, wherein a semiconductor component can be used, for example. However, the treatment surface can also designate a homogeneous material surface, which is tempered by several heating elements. For example, it may be preferable to use two or four heating elements in order to bring the treatment surface particularly homogeneously and quickly to the treatment temperature.

It is preferred that a control device can regulate the heating of the heating element in such a way that the treatment temperature is present at the treatment surface. This ensures optimum control of the treatment temperature and prevents undesirable overheating of the treatment surface.

In the sense of the invention, the control device is preferably an integrated circuit, processor, processor chip, microprocessor, microchip or microcontroller configured to regulate the temperature of the treatment surface by means of the at least one heating element according to predetermined treatment parameters. Such a control device is characterized by compactness, reliability, cost effectiveness, low power consumption and high control efficiency. In accordance with the invention, the control device is included in the device.

Preferably, the term heating element refers to the component which can be heated by the control device for example by applying an electric current. The at least one heating element is preferably a component for which various designs are sufficiently known from the prior art. For example, the heating element may comprise a power resistor, in which a well-defined temperature is generated depending on the current flow. A field effect transistor (FET) can preferably be used for quantitative control of the current flow through the heating element. However, it may also be preferable to use an FET itself as a heating element. In this case, the energy dissipation in the transistor is used to generate heat and to bring the treatment surface to the treatment temperature. FETs are particularly preferred as heating elements as they allow due to their small size of the device due their small dimensions. Furthermore, FETs are particularly reactive and ensure a particularly rapid response behavior of the heating elements due to a very dynamic heat generation and heat release.

Preferably, the control device can control what temperature is present at the treatment surface by setting the current feed to the heating element. For example, a calibration can be used to determine the correlation between the current flow and/or voltage at the heating element and the temperature at the treatment surface, so that a desired treatment temperature between 40° C. and 65° C. can be reliably ensured on the basis of the calibration. However, it may also be preferred to regulate the treatment temperature by the control device using a feedback loop. For example, it may be preferable to use a temperature sensor that measures the temperature of the treatment surface, wherein the control device regulates the current flow to the heating element on the basis of the temperature data. To this end the control device may, for example, include a microprocessor which allows to evaluate measurement data and set current parameters. This allows the temperature to be controlled very efficiently and reliably.

In the sense of the invention a microprocessor is preferably understood to be a data processing device, i.e. a processor, which is characterized by small dimensions in the range of a few mm and wherein preferably all components of the processor are present on a microchip or integrated circuit (IC). The microprocessor can be preferably also a microcontroller, which integrates further peripheral elements on the microchip besides the processor and comprises for example a data memory.

It is further preferred for the microprocessor to be installed on a printed circuit board (PCB). In addition to the microprocessor, preferable also has the heating elements and temperature sensors are installed on the PCB. This preferred embodiment allows for an extremely compact and robust construction of the device and a particularly intelligent temperature regulation by means of the microprocessor. The microprocessor is not only able to evaluate the measured temperature data and translate them into a control of the heating elements, but it can also rapidly and reliably take into account other parameters such as error messages or a user input into consideration.

In a preferred embodiment of the invention, the device is characterized in that the microprocessor and the heating element and optionally a temperature sensor are installed on a printed circuit board (PCB), at least the heating element and the temperature sensor being coated with a protective lacquer. In the sense of the invention, the protective lacquer is preferably understood to be a lacquer or paint which is intended to protect components of the PCB from environmental influences.

For this purpose, the protective lacquer is preferably electrically insulating and water-resistant. The property of the electrical insulation can be preferably quantified on the basis of the surface insulation resistance (SIR). The SIR may preferably be measured by leakage currents between the components of the printed circuit board. A high resistance corresponds to a good electrical insulation. Water resistance preferentially means that even at high humidity or in case of water penetration, the lacquered electronic components remain intact and no short circuit occurs. Water resistance can also be tested, for example, by measuring the SIR under conditions of high atmospheric humidity.

Numerous protective lacquers preferentially suitable for use are known in the prior art. Examples include protective lacquers based on acrylate, silicone or polyurethane. By applying the protective lacquer in the area of the heating elements and temperature sensors, these components are effectively protected against deposits, so that incorrect measurements of the temperature sensors can be avoided. On the one hand, this increases the accuracy with which a treatment temperature may be set and, on the other hand, it prevents overheating of the treatment surface due to an incorrect measurement of the temperature.

In a preferred embodiment, between 2 and 20, 2 and 10, preferably between 4 and 8 treatment programs are stored on the control device. Intermediate ranges can also be preferred, such as 4 to 20 treatment programs or even 8 to 20 treatment programs. The number of treatment programs has proven to be particularly advantageous. On the one hand, this allows the provision of a sufficient number of different treatments for an adaptation to the individual user or application. On the other hand, the number is small enough to avoid a confusing selection by means of the mobile device, thus increasing user-friendliness and compliance.

In a preferred number, the number of treatment programs corresponds to the product of a maximum number of a first selectable treatment parameter and a maximum number of a second selectable treatment parameter. A first selectable treatment parameter can be, for example, the treatment duration, while a second treatment parameter can be, for example, the treatment temperature. For example, if 2, 3, 4, 5, 6, 7, 8, 9 or 10 treatment temperatures in combination with 2, 3, 4 or 5 treatment durations are to be selectable, the apparatus will have a corresponding number of treatment programs (e.g. 4, 6, 8, 9, 10, 12, 14, 15, 16, 18, 20, 21 24, 25, 28, 30, 32, 35, 36, 40, 45 or 50).

In another embodiment, the device has only one operating element for heating the treatment surface according to the selected treatment program and the treatment program cannot be selected manually on the device itself. Due to its simple design, also this version is very popular with users. Incorrect settings due to differing operating elements, which cannot be explained sufficiently on the device itself, are effectively avoided.

In addition, the embodiment is characterized by a particularly compact and robust construction. Especially when transporting the device the risk of incorrect settings or wear and tear increases for example with the number of operating elements. These risks can be reduced by using only one operating element, which is preferably designed to be particularly robust against mechanical influences.

In the sense of the invention, an operating element is preferably understood to be any element which allows an electrical signal to be generated or an electric circuit to be closed by manual actuation. For example, it can be a push button or operating button which, when pressed, closes an electrical circuit and automatically returns to its initial position after being released. Other electrical switches such as rocker switches, latching switches, rotary switches, etc. may also be preferred. Such switches are particularly robust and represent simple operating elements which prevent incorrect settings. The operating element can also be formed by a capacitive sensor. For example, it can be a touchpad, a capacitive membrane keyboard or other capacitive sensor. It is well known that these touch sensors detect the touch of a finger or a hand based on the change in capacitance of a single capacitor or capacitor system. Advantageously, the sensors may be applied flat on the device, allowing for straightforward manual operation to trigger the treatment. The device is preferably configured in such a way that after manual actuation of the operating element a treatment cycle is initiated, i.e. the control device regulates the temperature of the treatment surface according to a selected treatment program.

Advantageously, no connection with a mobile device is required. Instead, the device may operate completely autonomously with regard to the actual performance of the treatment.

To provide a stand-alone device, i.e. one that can be used independently of a mobile device, it is particularly preferable to provide an operating element directly on the device. In some designs, however, it may also be preferred to use the input options of the mobile device as an operating element. These include, for example, a capacitive surface sensor, preferably a touch screen, as well as a microphone of the mobile device with corresponding downstream processing by the processor unit of the mobile device. In this case, the operating input for triggering the treatment may be for example a touch input on the touch screen of the mobile device or a voice command to the mobile device. The operating input is transmitted to the device through the interface, so that for a treatment purposes the device should be connected to the mobile device, e.g. by a plug connection.

In a preferred embodiment, the device is configured so that, independently of any existing communication to a mobile device, when actuated a last selected treatment program is executed.

Accordingly, the control device is preferably configured in such a way that one treatment program is always considered to be currently selected. When a connection to a mobile device exists, the mobile device can select a new treatment program via the interface. As soon as the selection has been made, that treatment program is defined as selected. Even if the connection is subsequently terminated, the device may still carry out a treatment using this last selected treatment program.

The device is therefore advantageously self-sufficient and does not require the connection or proximity of a mobile device. This allows for a particularly flexible use of the device when travelling or for leisure since the mobile device has not to be carried along. The treatment may therefore be in any situation carried out rapidly and on site without the need for a configuration or confirmation by means of a mobile device.

In a preferred embodiment, the interface and the control device are configured in such a way that a selection of one of at least two treatment programs by means of a mobile device is only allowed if an authentication has been successful. The mobile device may be preferentially recognized and authenticated, which is made possible in particular by assigning a unique identifier. For example, it may be necessary for the mobile device to transmit in addition to data specifying the selection of a treatment program an identifier in order to have the device accepting the corresponding selection. Authentication may also preferentially be carried out by cryptographic means known to the person skilled in the art, such as those used in public key infrastructures. Digital certificates and/or digital signatures may also be used. Such authentication can also prevent unauthorized external access to the device. For example, it could furthermore ensure that a thief cannot access the device with an arbitrary mobile device after the device has been stolen.

In a preferred embodiment, the device is characterized in that the selection of treatment programs by the mobile device is carried out wireless via the interface.

A wireless transmission of signals via the interface can, for example, take place in the form of electromagnetic waves. For this purpose, the interface would preferably comprise at least one receiving unit and/or one transmitting unit. A person skilled in the art knows how to equip the interface with suitable transmitters and/or receiving antennas, drivers and data converters, as well as which interconnections between the interface and other electrical elements of the device are necessary to ensure a wireless transmission. For example, a wireless transmission may take place via Bluetooth technology, whereby the interface has a corresponding Bluetooth module. Wireless transmission via Wi-Fi may moreover be provided, wherein the device exhibits a corresponding WLAN radio interface.

By means of wireless transmission, a selection of the treatment program by the mobile device may be carried out in a particularly convenient manner.

In a preferred embodiment, the treatment temperatures specified in the at least two treatment programs are between 40° C. and 60° C., preferably between 45° C. and 53° C., and particularly preferably between 47° C. and 53° C. These temperature ranges have proven to be particularly effective both for the treatment of itching, for example after insect bites, and for the treatment of herpes.

Even if the treatment programs are adapted to different applications or user preferences, it is therefore especially preferable to select the treatment temperatures of the individual treatment programs from the above-mentioned ranges.

In a further embodiment, the treatment temperatures are between 42° C.-56° C. The performance regarding the treatment of itching was surprisingly increased by the temperature range provided in this embodiment.

In a further embodiment, the treatment temperatures are between 42° C.-53° C. Thus, additional means for the treatment of itching can be provided.

In a further embodiment, the treatment temperatures are between 43° C.-48° C. The effectiveness of these treatment temperatures in the treatment of herpes is surprisingly high.

In a further embodiment, the treatment temperatures are between 47° C.-53° C. This temperature range has proven to be particularly effective and popular with the subjects. This was fortunate, because from a wide range of parameters, a range could be selected that was most effective against itching.

In another embodiment, the treatment temperature is selected from 50° C.-55° C., 55°-60 or 60° C.-65° C. These temperature ranges have been chosen contrary to the trend in scientific technology and can still provide effective relief in some subjects.

In another embodiment, the treatment temperature is maintained for a treatment period of 1-12 sec, preferably 1-10 sec, 3-5 sec, 4-6 sec. The efficiency of the device may be increased by said short treatment duration, which at the same time prove particularly effective.

In another preferred embodiment, a first of the selectable treatment programs specifies a treatment temperature between 48° C. and 53° C., while a second of the selectable treatment programs specifies a treatment temperature between 45° C. and 48° C.

The provision of at least two treatment programs with the above-mentioned parameters allows to provide optimal treatment programs for frequently occurring groups of subjects. It was observed that subjects can often be divided into two groups with regard to a perception of treatment temperature. One group feels that treatment temperatures above 48° C. are too painful, while another group reports particularly strong relief of itching at temperatures above 48° C.

In another preferred version, a first of the selectable treatment programs has a treatment duration of 1-3 seconds, while a second of the selectable treatment programs has a treatment duration of between 3-12 seconds. It was also observed that with regard to the treatment duration, different subjects preferred in particular two ranges. For some, a short treatment duration, optionally at higher treatment temperatures, is more comfortable, while other subjects describe longer treatment durations, optionally at low treatment temperatures, as particularly effective.

In a further preferred embodiment, the device comprises between 6 and 24 treatment programs, preferably 15 treatment programs, wherein the treatment programs differ in at least the treatment temperature or duration and wherein the treatment programs preferably allow the selection of any combination of 4 to 6, preferably 5, different treatment temperatures with 2 to 4, preferably 3, different treatment durations.

In a preferred embodiment, the selectable treatment programs cover a treatment temperature between 45° C. and 53° C., preferably between 47° C. and 53° C., and particularly preferably between 47.5° C. and 51° C.

In a preferred embodiment, the selectable treatment programs cover a treatment duration between 1 second and 12 seconds, preferably 2 seconds and 6 seconds, particularly preferably 3 seconds to 5 seconds.

In preferred embodiment, the coverage may correspond to an equidistant or essentially equidistant coverage. For example, the treatment programs may be selected to allow any combination of a preferred combination of a temperature range of 47.5° C. to 51° C. with a treatment time of 3 to 5 seconds, preferably any combination of a treatment temperature selected from the group consisting of 47.5,° C., 48° C., 49° C., 50° C. and 51° C. with a treatment time selected from the group consisting of 3 seconds, 4 seconds and 5 seconds.

The preferred treatment programs described above have proven to be advantageous both in terms of a high user-friendliness and from a therapeutic point of view in adapting to the individual requirements of different subjects. It has been observed that the selection of, for example, 15 treatment programs in the above-mentioned ranges ensures, on the one hand, a sufficient degree of individualization to achieve the best treatment results for broad sections of the population. On the other hand, the end user is not overloaded with an excessive number choices, some of which may be disadvantageous or even harmful. Instead, all specified treatment programs (i.e. a specified combination of treatment parameters) are preferably based on sound study results, with the possibility of a deliberately limited individualization to further increase patient compliance and treatment success.

Advantageously, the mobile device allows for an easy selection from the various treatment programs, whereby the device itself can be kept slim and simple and as described above may for example have a maximum of one operating element for executing the selected treatment program.

In a preferred embodiment of the invention, the size of the treatment surface is less than 1 cm², preferably between 20 and 80 mm².

These sizes of treatment surfaces have proven to be particularly beneficial for treating itching after an insect bite or for treating herpes.

In the case of herpes diseases, especially in the mouth (so-called herpes labialis), a maximum size of the treatment surface of 60 mm² is ideal to cover all possible affected areas. In particular, a treatment surface between 20 and 50 mm² is suitable to cover all typical affected areas of skin when the device is applied only once. Furthermore, a herpes treatment device which has such a treatment surface can be kept particularly compact.

For the treatment of insect bites, especially mosquito bites, the size of the treatment surface is preferably between 10 mm² and 100 mm², especially preferred between 20 mm² and 60 mm², most preferred between 30 mm² and 50 mm². These sizes are suitable for a target treatment of the entire affected site without heating unnecessarily unaffected skin areas.

In addition, such devices of such size are particularly compact and may be kept at the size of a lipstick. Such a compact device is readily and willingly worn permanently on the body or in a carry-on bag such that a treatment may be carried out at any one time. This considerably increases the success of the treatment.

The treatment surface is preferably round, a shape which is particularly suitable for the treatment of herpes or insect bites, whose affected skin areas often have an almost round shape. However, any shape can be used, for example polygonal or convex shapes, which prove suitable for the treatment of herpes or insect bites. For example, a shape of a lipstick may be employed, in which case the user is encouraged to press the device lightly during the treatment. Said embodiment may trigger a psychological effect enhancing the subjective well-being during a treatment. The amount of heat transferred may moreover be improved. An organic form can also be used, which proves particularly suitable for treating the lips or other areas of skin.

In a preferred embodiment, the treatment surface exhibits the shape of a rounded rectangle. For example, the longer side of the rectangle may exhibit a length between 5 mm and 12 mm, preferably 6 mm to 10 mm, more preferably 7 mm to 9 mm, while the shorter side of the rectangle may exhibit a length between 2 mm and 8 mm, preferably 3 mm to 7 mm, more preferably 4 mm to 6 mm. In particular for the provision of very compact devices the above-mentioned shapes and sizes have proven to especially advantageous. On the one hand, sufficient contact is assured with affected skin areas. On the other hand, the entire device may be designed flat, for example similar to a USB stick, wherein the shape of the housing follows especially in the front treatment area the shape of the treatment surface.

In a preferred embodiment, the heating element heats the treatment surface to a treatment temperature between 42° C. and 56° C., preferably between 45° C. and 53° C., and particularly preferably between 47° C. and 53° C., and the treatment temperature is maintained for a treatment time of 1 sec to 12 sec, preferably 1 to 10 sec. The aforementioned ranges for treatment parameters are particularly suitable for the treatment of itching after an insect bite, such a mosquito bite.

Pain and other unpleasant sensations such as tension or itching can be surprisingly effectively relieved. This is especially the case after an insect bite, for example a mosquito bite. The heat impulse causes nerve stimulation, which greatly reduces the subjective perception of pain or itching in the affected areas. Surprisingly, the heat transfer results in a masking of the unpleasant sensation by other temperature-dependent skin sensations. Unlike conventional methods for the treatment of itching, this method additionally aims at a regulation of the pain receptors and activates the free nerve endings of the C fibers by means of the heat treatment. The knowledge about the mechanism of action in itching is thus exploited to regulate the sensory perception triggered by the fibers in a surprising manner by means of heat treatment.

Furthermore, it was recognized that the combination may achieve a particularly strong masking of unpleasant sensations, since the thermal and capsaicin receptors TRPV1 and TRPV2 are simultaneously activated locally at the affected sites. A person skilled in the art would not have assumed, even knowing literature, that the activation of these receptors allows for the observed particularly effective masking of the unpleasant sensations.

The treatment temperatures between 42° C. and 56° C., preferably between 45° C. and 53° C., and more preferable between 47° C. and 53° C. in particular in connection with a treatment duration of 1 sec to 12 sec, preferably 1 to 10 sec are especially preferred for applications against itching after insect bites, so that these are especially preferred for a treatment surface of 10 mm² and 100 mm², more preferably between 20 mm² and 60 mm².

In a preferred embodiment, the heating element heats the treatment surface to a treatment temperature between 42° C. and 53° C., particularly preferably between 43° C. and 48° C., and the treatment temperature is maintained for a treatment time of 1 sec to 12 sec, preferably 1 to 10 sec, more preferably between 2-5 sec. The above-mentioned ranges for treatment parameters are particularly suitable for the treatment of herpes diseases.

Studies have shown that the risk of combustion at a temperature level of 44° C. to 51° increases by a factor of two with every degree Celsius. In addition, it was realized that a thermolability of the DNA-binding protein ICP8 may be exploited to effectively prevent replication of the herpes virus DNA. Studies have shown a reduction of the protein's binding activity to the viral DNA by about 50% at a temperature of 45° C.

On the one hand, thus a temperature, which is as high as possible, should be chosen for the application of heat against herpes, on the other hand, the associated pain may lead to premature discontinuation of the treatment and impede a treatment success.

By selecting a temperature range of 43-48° C. for the treatment phase of 1-10 sec, excellent results in terms of a decrease in herpes blisters may be achieved within a few days. Notably, without complaints on a stabbing pain. Compliance and therapy success are consistently good for these parameters.

The above-mentioned temperature ranges are also particularly preferred in combination with a treatment surface of preferably between 10 mm² and 80 mm², especially between 20 mm² and 50 mm², which are preferred for herpes diseases.

In another preferred embodiment, the size of the treatment surface is between 1 cm² and 18 cm², preferably between 6 and 9 cm².

This embodiment is particularly suitable for the treatment of large sites of skin affected by itching, which may occur in case of chronic pruritus, allergies or jellyfish stings.

It has been recognized that with a larger treatment surface of about 1 cm² to 18 cm², preferably of at least 2 cm², at least 3 cm², at least 4 cm², more preferably between 6 cm² to 9 cm² and most preferably between 7 cm² and 9 cm², large surfaces of skin affected by pruritus may be treated in a surprising efficient manner. For instance, in case of skin rashes, it is possible to transform the itching sensation into a bearable pain sensation through heat transfer by simply and comfortably placing the treatment surface on the corresponding skin areas. Secondary damage to the skin, such as wound formation due to severe scratching, may as a consequence be effectively avoided. With prior art devices the treatment of large areas of skin would require repeated application at different positions. However, due to a time delay between the applications an effect to the same extent cannot be achieved.

It may also be preferred to use a treatment surface of at least 6 cm² or at least 7 cm² in size.

External stimuli of a chemical, mechanical or physical nature that can trigger itching are perceived by three different receptor cells (sensory cells). These sensory cells involve what are called open nerve endings, whose structures perceive irritations in the epidermis and the underlying dermis, and whose axons conduct of perceived irritations to the spinal cord. The non-myelinated C fibers are of particular importance in these sensory cells. Their receptive structures are found partially up to 0.1 mm below the skin surface. C fibers are divided into polymodal mechanically and heat-sensitive fibers and mechanically insensitive C fibers, which can also be stimulated by heat. C fibers perceive not only pruritogenic stimuli, but also serve as nociceptors (pain receptors). It is shown in the literature that heat receptors as counter receptors may suppress the sensation of itching. The individual C fibers perceive stimuli of a specific area of the skin, whereby a defined area of skin is innervated by a sensory cell. This region is called the receptive field. The receptive fields of C fibers can partially overlap. Studies on human beings through what is called micro-mapping discovered surprisingly that the mechanically insensitive C fibers have receptive fields of up to 5 cm² in size; the C fibers, which are mechanically sensitive, are somewhat smaller, up to 2 cm² in size. Surprisingly, it was found that with a preferred treatment surface size of at least about 6 cm² or at least about 7 cm², receptive fields of both types of C fibers can be addressed in a particular efficient manner. Thus, with the preferred treatment sizes between 7 cm² and 18 cm² the receptive fields of the different types of C fibers are covered surprisingly well and, in addition, the effect of the horizontally outflowing heat is compensated for. Thereby itching, even in affected skin areas smaller than the treatment surface, can surprisingly be very effectively treated.

In a preferred embodiment, the device includes at least one energy storage device.

Energy storage may be realized for example by means of at least one accumulator, a battery and/or a capacitor. Energy storage preferably refers to an electrical energy storage. A battery is an energy storage, preferably an electrochemical energy storage, whose stored energy may preferably not be recharged after usage. Examples include zinc-manganese batteries, alkaline-manganese batteries, zinc chloride batteries, zinc-carbon batteries, zinc-air batteries, mercury oxide-zinc batteries, silver oxide-zinc batteries, nickel oxyhydroxide batteries, lithium batteries; lithium iron sulphide batteries, aluminium-air batteries, bio-batteries, e.g. based on magnesium/NaCl/iron+molybdenum+tungsten and/or Edison-Lalande elements. Batteries may also include button cells batteries.

A capacitor is preferably an electrical component that stores electrical charge in an electric field.

In particular, the energy storage device may be at least one accumulator.

In contrast to batteries, rechargeable batteries or accumulators are preferably rechargeable. Accumulator may include for example lithium ion batteries, lithium cobalt dioxide batteries, lithium polymer batteries, lithium manganese batteries, lithium iron phosphate batteries, lithium iron yttrium phosphate batteries, lithium titanate batteries, lithium metal polymer batteries, lithium air batteries, lithium sulphur batteries, sodium nickel chloride high temperature batteries, Sodium-sulphur batteries, sodium-ion batteries, nickel-cadmium batteries, nickel-iron batteries, nickel-hydrogen batteries, nickel-metal hydride batteries, nickel-zinc batteries, lead acid batteries, PTMA batteries, rechargeable alkaline manganese batteries, tin-sulphur-lithium batteries, silver-zinc batteries, vanadium redox batteries and/or zinc-bromine batteries, silicon air batteries. Lithium-polymer and/or a metal-hydride battery may also be used, which are particularly flexible, adaptable to the applications and particularly powerful.

The aforementioned embodiment of the device has the advantage that the device is supplied with electrical energy both when the interface is connected to a mobile device and also independently of such connection.

In particular the embodiment comprising at least one accumulator (or possibly a capacitor) may be supplied with electrical energy and charged when the interface is connected to a mobile device.

The accumulator may be dimensioned in such a manner that it allows for the provision of energy for at least one application, preferably at least 10, 20, 30, more preferably of at least 40 applications. Thereby, the device does not necessitate a connection to the mobile device for use and proves particularly flexible.

In a preferred embodiment, the accumulator includes a solid state accumulator, preferably a lithium ceramic accumulator. Said accumulator is distinguished by a particularly high safety level and preferably is even usable after partial destruction.

In all the above-mentioned embodiments, the device can be kept compact and directly plugged to the mobile device, such as a smartphone, via an interface. In this case, a user can use the device through his smartphone, for example by mounting the device onto the smartphone, whereby a mechanical connection is preferably created by the coupling interfaces themselves. The smartphone may be used to control and/or handle the device. In the case of wireless interfaces, the device itself may be shaped in such a way that it may be mechanically connected to the mobile device, for example by plugging it onto the mobile device, and therefore a connection is also possible without a wired interface.

Advantageously the device itself can be kept particularly compact. For example, it is possible that the device has a width of 15 mm to 25 mm, a thickness of 10 mm to 20 mm and a length of 15 mm to 30 mm. On one of its sides the device may have a physical interface for the mobile device and on the opposite side a treatment surface. The interface may have a connector, for example, for a Lightning or USB type C connections. The choice of interface is preferably based on the connection options available at the mobile devices. The length preferably relates to the distance between interface and treatment surface. Width and thickness denote orthogonal directions, wherein it is preferred that the slightly larger width corresponds to the dimension which, when in the attached state, corresponds to the width of a flat mobile device.

When attached to the mobile device it is preferred that the mobile device may be used to select the treatment programs, as well as to supply power to the device. To this end a software (‘app’) customized for this purpose may be provided on the mobile device. It may also be preferable to omit having a separate operating element on the device itself and instead trigger the treatment by the app such that the touch screen of the mobile device may function as an operating element. In any case, the device is advantageously configured in such a manner that the mobile device enables a selection of the treatment programs stored on the device (for example within the firmware) without the mobile device or the app being able to change the operating parameters. Thereby, a device may be provided, which is conveniently operated by means of the mobile device, but which does not compromise safety standards. Advantageously, the external power supply allows for particular small dimensions.

However, it is particularly preferred that the device is self-sufficient and comprises its own compact energy storage. It has been recognized, that in contrast to a direct coupling to a mobile device, an improved handling is achievable. An affected skin area can be targeted much easier with the help of a compact but self-sufficient device than with a device that is attached to a mobile device. In the latter case, the mobile device partially obstructs the view, whereas in the former case the dimensions of a device may be chosen in such a manner that the device fits comfortably in a hand without obstructing the view.

In a preferred embodiment the device may have, for example, a width of 15 mm to 25 mm, a thickness of 10 mm to 20 mm and a length of between 50 mm and 100 mm, preferably 60 mm to 80 mm. By integrating an energy storage device, a stand-alone device may be somewhat longer in size, than the previously described mountable version. However, a stand-alone device (see FIG. 1) is particularly easy to handle and guarantees a precise placement of the treatment surface on the skin area to be treated.

For a communication with the mobile device, the device may have a wireless interface, e.g. a Bluetooth interface. Preferably, a treatment program is selected via the interface using a mobile device. For this purpose, the mobile device may be equipped with a software (‘app’) customized for this purpose. The selected treatment program is preferably executed after an operating element on the device has been operated. It is preferred that the last selected treatment program is executed when the operating element on the device is operated. The device can thus be used without connection to a mobile device.

In a particularly preferred embodiment, the device comprises a head element and a body element, which are pluggable connected to each other via an interface, preferably the treatment surface and control device are situated in the head element and the operating element and an energy storage device are located in the body element. The interface is preferably a plug-in connection which, in addition to the functionality described in relation to the interface, such as, for example, data exchange and/or energy supply, moreover, ensures mechanical stability. Preferably, the interface between the head element and the body element can simultaneously serve as an interface for a mobile device. It is preferred that the treatment surface in the head element is located on the side opposite the interface (or a body element connected to it). Such a two-part device combines a number of advantages of the previously described embodiments.

On the one hand, an extremely compact head element can be mounted onto a mobile device. To this end the head element is preferably separated from the body element and connected to a mobile device via the available interface. The interface now functions as an interface to the mobile device, so that in the connected or mounted state, both a selection of the treatment programs and a power supply by means of the mobile device are possible. To this end a corresponding software (‘app’) may be provided on the mobile device. However, advantageously the mobile device does not allow for making any safety-relevant changes to the treatment programs. Instead also in this embodiment, the mobile device may preferably only select the treatment programs that are stored on the control device of the head element (e.g. within the firmware).

On the other hand, the device may also be used advantageously in a self-sufficient manner, i.e. independent of a mobile device. This is preferably achieved in an assembled state, i.e. in a state in which the head element and body element are connected via the interface. The energy supply is preferably provided by the energy storage situated within the body element. By activating the treatment program, the last selected treatment program preferably executed. Preferably said treatment program corresponds to the treatment program that was selected during a connection of the head element with the mobile device.

Furthermore, it may also be preferred that the two-part device exhibits two interfaces for a connection to a mobile device. On the one hand, the interface between the body and head element may preferably be used as a first interface to a mobile device. On the other hand, the device may preferably have another, preferably wireless interface, for example a Bluetooth interface for a second interface with the mobile device. The wireless second interface enables the device to be connected to the mobile device even during an assembled state, i.e. in a state in which the head element is connected to the body element via the interface, so that the interface is not available for connection to a mobile device. The additional, preferably wireless interface may be used as described, in particular for selecting a treatment program or for further data exchange.

In a preferred embodiment, the device may comprise a cap to protect the interface on the head element when disconnected. Preferably the cap as well as the body element and the head element are dimensioned in such a manner that the cap may be positioned on the end of the body element facing away from the interface and at the same time may not be lost.

In a preferred embodiment, the head element exhibits a width of 15 mm to 25 mm, a thickness of 10 mm to 20 mm and a length of 15 mm to 30 mm.

In a preferred embodiment, the body element exhibits a width of 15 mm to 25 mm, a thickness of 10 mm to 20 mm and a length of 40 mm to 80 mm.

In a preferred embodiment, the device comprising a head element and a body element, which are connected via an interface, exhibits a width of 15 mm to 25 mm, a thickness of 10 mm to 20 mm and a length of 60 mm to 120 mm, preferably 70 mm to 100 mm.

In a preferred embodiment of the invention, the interface is configured in addition for a powers supply by means of a mobile device.

To this end it is preferred to an enable energy transfer from the mobile device to the device via the interface. The energy transfer relates in particular to the transfer of electrical energy. However, the energy can also be transferred in another form and subsequently be converted into electrical energy by the device. The transfer, in particular of electrical energy, can take place via cable. In this case, the interface is formed by providing the possibility of a cable connection between the mobile device, or its interface, and the interface of the device.

However, the transfer may also take place in a wireless manner, wherein at the side of the device the suitable interface for this purpose preferably consists of an energy converter which converts wirelessly transmitted energy into electrical energy to be used to supply the device with power.

The transmitted electrical energy received at the interface is preferably transferred to components of the device which consume electrical energy, such as the heating element or the control device. This transfer may be realized by suitable connections, for example by cables. An energy storage device may be connected in between and/or downstream. The use of suitable voltage regulators and/or charge regulators to supply and/or charge the energy storage device and the electrical connection of individual components of the device to the interface is also preferred and may be routinely carried out by a person skilled in the art.

Preferably the transfer of energy and data may occur in parallel. Some interfaces, such as USB, provide for a parallel voltage and data transfer. Even with a wireless interface, electrical energy and/or data may be transmitted, preferably in parallel, for example by means of induction coils or by means of electromagnetic radiation, which is converted into electrical voltage by solar cells and/or photodiodes.

In a preferred embodiment, the device is configured for a power supply and/or charging of the energy storage by means of wireless energy transmission, preferably via the interface.

In a wireless energy transfer, electrical energy is preferably transferred without a connection between the device to be charged and the mobile device, which is based on an electrical cable. The electrical energy can be transferred by electromagnetic fields, for example. Inductive coupling through at least one coil each in the mobile device and in the device may be used to this end. It may also be advantageous to capacitively couple the device and the mobile device through at least one capacitor plate each. Furthermore, over long distances a far-field transmission may preferably be used to transfer electrical energy contained in the electromagnetic waves, which are transferred between a transmitter and a receiver.

Similarly, light emitted by the mobile device may transfer energy that is converted into electrical voltage by means of at least one solar cell located on the device. It may also be preferable to transfer electrical energy directly between conductive surfaces of the mobile device and the device via a contact existing between these surfaces. The transferred energy may be used directly to heat the heating element or preferably to charge a battery integrated within the device.

A person skilled in the art would know that for this purpose the device has to be equipped with suitable elements, including for example at least one induction coil, a capacitor plate, an antenna, a solar cell, a photodiode, a charge controller and/or a voltage regulator and how to interconnect the suitable elements in order to implement the wireless transfer of energy.

By means of wireless energy transfer, tedious cabling between the device and mobile device may be avoided.

In a preferred embodiment, the device is configured for a power supply and/or a charging of the energy storage device by a cable, in particular via the interface. In this embodiment, the device, in particular the interface, comprises a cable or a plug and/or a socket for connection to a cable. Furthermore, it is preferred that the electrical energy within the device is transferred to suitable components, in particular to the energy storage by means of connections, for example. A voltage regulator and/or charge regulator is advantageously interposed. A person skilled is aware of suitable components and their interconnections. Transfer of electrical energy in this manner is particularly robust, energy-efficient and cost-effective.

In a preferred embodiment, an interface is selected from the group comprising: Bluetooth, Lightning, Jack plug, Coaxial plug, Apple 30-pin dock connector, ASUS Media Bus proprietary, CAMAC, EISA, ISA, LPC, MBus, MCA, Multibus for industrial systems, NuBus or IEEE 1196, OPTi local bus, PCI, ATA, PATA, IDE, EIDE, ATAPI, S-100 bus or IEEE 696, SBus or IEEE 1496, SS-50 bus, Runway bus, GSC/HSC, Precision Bus, STEbus, STD Bus, Unibus, Q-Bus, VLB or VL-bus, VMEbus, PC/104, PC/104-Plus, PCI-104, PCI/104-Express, PCI/104, Zorro II and Zorro III, 1-Wire, HyperTransport, I²C, PCIe, SATA, SPI bus, UNI/O, SMBus, IrDA, WLAN, ZigBee, NFC, Wibree, WiMAX, IrDA, optical radio relay, eBus, USB, Micro USB, Type C and/or FireWire.

By providing an interface selected from said group, a high degree of flexibility with regard to the mobile device used and the available interface is provided for. Furthermore, these interfaces have proven their suitability for a wide range of different applications. A person skilled in the art knows how to design such an interface. For example, a person skilled in the art would know that he would have to install a suitable socket and/or plug for a Lightning interface, and a person skilled in the art would also know that he would have to use suitable processors, e.g. host controllers. Moreover, a person skilled in the art would know how to connect the electrical components of the device to the interface.

In the case of a WLAN interface, for example, the person skilled in the art would also know that he would have to install in the device at least one suitable antenna for sending and/or receiving signals. The selection of suitable control processors, data converters and/or controllers as well as an interconnection of the interface within the device is routinely feasible for a person skilled in the art.

It may be preferable to use the interface to connect the device directly to an electrical outlet for a power supply and/or charging. In a preferred embodiment, it is possible, for example, to draw electrical energy directly from a power socket using a standard USB charger.

In a preferred embodiment, the interface is additionally configured for a data exchange with a mobile device.

Data preferably relates to information that can be output, received and/or processed by an electronic data processing device. A data processing device is preferably a device selected from the group of computers, microcomputers, processors, microprocessors, integrated circuits, smartphones, other mobile devices and/or control devices. Preference is given to data in digital format, especially in bits. However, analog data are also conceivable. Likewise, data can preferably be stored in a memory or on a storage medium.

Data transfer with a mobile device preferably refers to the exchange of data with the mobile device. Data can preferably be received and/or sent by the device. Likewise, data can preferably be stored and/or processed by the device.

In order to be configured for data transfer via the interface, a data line, preferably several parallel data lines, should be established between the device and the mobile device via the interface.

This means that a transfer between mobile device and interface as well as between interface and other electrical components of the device is enabled via data lines. The transfer between mobile device and interface can preferably take place via an interface of the mobile device, which, depending on the interface, may be wired and/or wireless, as described above. The transfer between the interface of the device and other components may for example be realized by physical signal lines in form of cables, which enable the transfer of electrical signals.

It may be preferred that at least one of the elements of the mobile device, the mobile device interface, the device interface and the electrical components of the device uses a different data format than at least one of the other elements of this group. It may therefore be preferred that at junctions of elements, which use different formats, at least one data converter is present which converts the data in the direction of transfer into a data format suitable for the following element. If such a data converter is assigned to the interface of the device and/or follows in the direction of the components of the device, said at least one data converter is preferably part of the device.

For transmitting (sending) data, the device and/or its interface preferably comprises at least one data transmission (sending) device. This can send data from the device via the interface in the direction of the mobile device. The data can preferably be generated by a control device integrated in the device and/or be present on a data memory of the device. Preferably, the data sending device can also act as a data converter or comprise such a converter. A person skilled in the art knows suitable devices and/or can set such device up routinely.

For receiving data, the device preferably comprises at least one data receiving unit. The unit can receive data coming from the mobile device via the interface, convert the data, if necessary, into a suitable data format and, for example, forward the data in a suitable manner to a control device for further processing and/or a storage medium for storage. A person skilled in the art knows possibilities to implement suitable units routinely.

It may also be preferable for the data sending and receiving unit to be combined in a common unit.

As described above, the device preferably includes a memory that can be used for data transfer. This can be, for example, a memory selected from the group comprising solid state memory, RAM memory, ROM memory, EPROM memory, EEPROM memory, flash memory and/or other memory technologies.

The data can be transferred in parallel and/or serially. In a parallel transfer, several digital information units, which are referred to as bits, can be transferred simultaneously.

It is particularly preferred that the data is transferred from the device to the mobile device. The data can be, for example, an indication of the currently selected treatment program and the treatment parameters defined, such as the treatment temperature, the treatment duration and/or a time sequence of treatment temperatures to be applied.

Preferably, the selected treatment program can be displayed on the mobile device so that the user always receives a feedback on whether the selection was made correctly.

The data can also include measured values from the device, for example from a temperature sensor, so that the course of treatment and the heating of the treatment surface can be tracked in real time on the mobile device. The functionality is particularly popular with test persons and increases user compliance.

The data transmitted by the device can preferably also be linked to user data. In doing so, further relevant metadata can be generated, for example, treatment success, healing process, frequency of use, etc.

In addition, the mobile device, which is for example a smartphone, can generate further data, such as GPS position data, time stamps, photos of itchy skin areas etc. These can all be linked and/or correlated with each other, which allows the generation of statistically relevant data, e.g. concerning successful treatment therapies or a geographically and/or temporally limited occurrence of an insect plague.

In a preferred embodiment, the mobile device is selected from the group comprising PC, laptop, desktop PC, mobile phone, smartphone, tablet computer, personal digital assistant (PDA), notebook, subnotebook, walkman, discman, MP3 player, pocket TV (portable TV), e-book reader, portable electronic media output device, GPS device, portable satellite communication interface device, handheld, pocket computer (‘pocket computer’), mobile computer, camera, video camera, wristwatch, calculator, television, MacBook, iPhone, iPad, iPod, iMac, Mac mini, Mac Pro, Smartwatch and/or Powerbank.

These mobile devices have proven to be particularly practical for use with the device. Especially mobile devices which have an inherent high computing power and a high flexibility regarding their application may form a particularly powerful system in connection with the device. Such devices are represented for example by smartphones. In case of smartphones, surprising advantages can be achieved through application programs (‘apps’) individually tailored to the device. For example, networked user data can be used to allow for statistically relevant statements on successful treatments, which, if a sufficiently large number of data to be evaluated is available, may go far beyond the statistical significance of an individual medical study. For example, synergistic effects may be achieved by predicting insect plagues based on user data obtained from the past, since the device may not only treat insect bites, but also aid to prevent them. Furthermore an improvement of a medical treatment of itching by a specialist doctor is possible, which is based on the evaluation of individual user data.

In a preferred embodiment of the invention, the device comprises at least one temperature sensor for measuring the temperature of the treatment surface, wherein the control device regulates the at least one heating element based on the measurement data of the temperature sensor.

In the sense of the invention, a temperature sensor is preferably an electrical or electronic component which generates an electrical signal depending on the temperature at the sensor. Many temperature sensors are known in the prior art, such as semiconductor temperature sensors, resistance temperature sensors, pyroelectric materials, thermocouples or oscillating crystals.

The control device is also preferably configured such that it can receive and evaluate the measured values of the temperature sensors in order to regulate the heating plates. The regulation of the heating plates can preferably be accomplished by applying an electric current or voltage.

It is particularly preferred that the temperature sensor measures the temperature of the treatment surface directly, i.e. that the temperature sensor is in contact with the treatment surface, wherein the temperature sensor may be present on both the internal side of the treatment surface and the external side of the treatment surface or be implemented within the treatment surface. However, it may also be preferred that the temperature sensor does not directly contact and monitor the treatment surface, but instead, the heating elements or a material point between the heating elements and the treatment surface. In the case of several heating elements which heat the treatment surface, it may also be preferred to place the temperature sensor between the heating elements. Likewise, a conclusion may be drawn regarding the temperature of the treatment surface from the measured data for the temperature across the heating elements or from a measuring point at a certain distance from the treatment surface. In the sense of the invention it is preferred that the temperature of the treatment surface relates to the average temperature of the treatment surface. Especially preferred the treatment temperature relates to the average temperature of the external side of the treatment surface, while it is in contact with a skin during application of the device.

Evaluation of the temperature of the treatment surface allows a particularly precise regulation of the at least one heating element in order to ensure an optimal temperature distribution on the treatment surface and thus a heat transfer to the skin parts to be treated. Especially with regard to the manifold usage possibilities of the device for the treatment of different diseases which can be accompanied by itching, a temperature-based feedback regulation with the aid of the control device is suitable for performing a reliable hyperthermic treatment with optimal temperature values. Such a device for controlling the treatment temperature is particularly simple, robust and cost-effective.

In another preferred embodiment, the device includes a watertight housing. The housing is preferably an external casing for the device that encloses in particular the control device and other electronic components.

In the preferred embodiment, the housing is designed such that all housing cutouts, e.g. fo operating elements, interfaces, housing separations are watertight or insensitive to water. For example, sealing rings or suitable sealing elements, for example made of elastomers, can be used for this purpose. However, the person skilled in the art knows numerous other technical solutions for ensuring a watertight housing. The watertight design of the housing represents an (additional) safety element, as this can effectively prevent damage to the control device or other electronic components due to liquids entering the housing. The watertight housing also prevents corrosion and thus extends the service life of the device. Especially in combination with the use of a protective lacquer, safety can be increased synergistically. This is particularly important for disinfection processes of the device and especially the treatment surface. The device can thus be thoroughly disinfected with ease and without errors by preferably immersing or placing the entire device in a disinfecting liquid and leaving the device for a certain minimum period of time.

In other preferred embodiments, the device includes additional safety elements that control the temperature of the treatment surface.

In a preferred embodiment, the device includes a hardware-implemented temperature monitor that reversibly limits the maximum temperature of the treatment surface and a safety fuse that switches off the device in case of a short circuit or uncontrolled heating.

The maximum temperature preferably refers to the maximum temperature that the treatment surface reaches during the treatment phase. The hardware-implemented temperature monitor is advantageous in ensuring that the maximum temperature is not exceeded.

The “hardware-implemented temperature monitor” preferably refers to a temperature control system for the treatment surface, which can shut off the power supply of the heating elements for the treatment surface based on hardware. In particular, the “hardware-implemented temperature monitor” preferably allows to cut the power supply of the heating elements, when the maximum temperature is exceeded, independently of the regulation of the heating elements by the control device, e.g. the microprocessor.

If, for example, a firmware is installed on the control device to regulate the heating elements, it is preferred that the hardware-implemented temperature monitor reliably limits the maximum temperature of the treatment surface even in the event of a failure or incorrect performance of the firmware. Thereby it can be effectively ensured by simple design means that the treatment surface of the device does not exceed a maximum temperature. Even in the event of a failure of the control device, for example, after infiltration liquids, the hardware-based temperature monitor can advantageously ensure at all times that the treatment surface does not exceed a maximum temperature. The additional technical element for temperature monitoring makes it possible to maintain an excellent safety standard without interfering with the operation of the hyperthermic treatment device.

For example, a value between 54° C. and 58° C., preferably around 56° C., can be selected as the maximum temperature. In this case, the device may include a hardware-implemented temperature monitor which limits the maximum temperature of the treatment surface to a value between 54° C. and 58° C., preferably about 56° C.

In the sense of the invention, terms such as about, approximate, near or synonymous terms preferably denote a tolerance margin of less than ±10%, preferably less than ±5%, especially preferred less than ±1%.

Such a maximum temperature is especially preferred in connection with an embodiment in which the treatment temperatures stored on the control device are between 45° C. and 53° C., for example. In general the temperature is thus guided to a certain treatment temperature according to the selection of the treatment program, which should not exceed 53° C. If due to a failure of the control device this happens nevertheless, the hardware-implemented temperature monitor can reliably ensure that the maximum temperature of 56° C., for example, is not exceeded.

Other, suitable maximum temperatures, e.g. between 53° C. and 56° C. or lower, may also be preferred depending on the treatment programs envisioned.

By means of a hardware-implemented temperature monitor it can be ensured particularly effectively using simple design means that the treatment surface of the device does not exceed a maximum temperature. Even in the event of a malfunctions of the control device, e.g. after an infiltration with liquids, the hardware-based temperature monitor can advantageously ensure at any time that the treatment surface does not exceed a maximum temperature (e.g. a value between 54° C. and 58° C., preferably around 56° C.). This additional technical element for temperature monitoring enables to maintain an excellent safety standard without interfering with the operation of the hyperthermic treatment device.

As a further safety element, the device may have a safety fuse which interrupts the power supply to the device in the event of a short circuit of the device or uncontrolled continuous heating of the device. In the sense of the invention, a safety fuse is preferably understood to be an overcurrent protection device in which, for example, a circuit can be interrupted by the melting of a fuse conductor as soon as the current intensity exceeds a limit value over a time to be determined. It is preferred that the safety fuse is present in the device between the supply voltage being fed into the device and the device itself. If a malfunction should occur that is characterized by the flow of an uncontrolled high current from the supply voltage feed into the device, the safety fuse will advantageously shut down the power supply to the device completely. A safety fuse offers a sufficiently fast and extremely reliable protection.

It has been shown that even with faultless design of the device and the supplying of a hardware-implemented temperature monitor, it is not possible to rule out the occurrence of continuous heating of the heating elements in extremely rare instance due to incorrect operation or s damage to the device. Continous heating of the heating elements is preferably understood in the sense of the invention to mean that the temperature of the heating elements rises uncontrolled, i.e. without a temperature-based regulation with the aid of the control device. If the hardware-implemented temperature monitor fails in such cases, the treatment surface can rise uncontrollably to temperatures far above the desired maximum temperature, for example to temperatures in excess of 65° C.

Although such undesired continuous heating occurs very rarely, it may cause severe injuries to the subjects. This is especially due to the fact that the skin areas to be treated hyperthermally, such as the lips, are usually particularly sensitive and for example are characterized by redness, swelling or even wound formation. A temperature distinctly elevated temperature above 65° can lead to severe local pain and can cause burns to the skin at these sites.

In view of the special circumstances of the use of the device and the associated safety requirements, the safety fuse is particularly advantageous in order to guarantee that the heating of the treatment surface is shut down even in the most unlikely instance of a malfunction. For example with the aid of the safety fuse, independently of any temperature measurement, excessive heating of the treatment surface, due for example to defective temperature sensors, can be suppressed. It was recognized that the power supply to the device represents a central regulatory interface that meets the highest safety requirements. By integration of the safety fuse into the current flow for supplying the device it is possible to ensure that a maximum supply current will not be exceeded for a certain time. Since continuous heating and uncontrolled heating of the heating elements above the desired temperature are related to increased current flow, in this way overheating of the treatment surface can be avoided especially reliably. In particular, the current controller can react very quickly before the current is present for long enough that it will produce a temperature corresponding to its strength. A safety mechanism as a final means based purely on temperature may not be fast enough as a result of the thermal inertia of the components involved.

The combined use of a hardware-implemented temperature monitor and a safety fuse is particularly advantageous.

For example, one drawback of the safety fuse is that following the single triggering it permanently disconnects the supply voltage from the device Resumption of the use of the device following triggering of the safety fuse requires repair by a technician, for example replacement of the safety fuse. In terms of cost, the device has generally become unusable when the fuse has been triggered.

Advantageously, however, the hardware-implemented temperature monitor is set such that it does not need to cause permanent shutoff of the power supply to the device. Instead, the hardware-implemented temperature monitor is designed in such a way that if the temperature of the treatment surface exceeds a maximum temperature, the power supply to the heating elements is interrupted during the time period exceedance. Thus the current interruption by the hardware-implemented temperature monitor is advantageously reversible, i.e., as soon as the temperature of the treatment surface again drops below the maximum temperature, the heating elements can heat again.

Thus even after a one-time occurrence of a malfunction the normal use of the device can be continued. The user would also not notice the malfunction, since as a result of the maximum temperature selection, the effectiveness and the independence of the temperature controller, no temperatures perceived by the user as unpleasant will develop and once a malfunction has occurred, the device can function perfectly again upon the next use.

The combination of the safety features of a hardware-implemented temperature monitor with a safety fuse allows for surprisingly reliable control of the temperature by the most economical means possible because of the hierarchy of safety barriers. Such a combination of additional safety barriers is particularly useful for a device that is connected to a mobile device.

A further synergistic effect of combining the safety features of a hardware-implemented temperature monitor with a safety fuse can be seen in the fact that an unlikely but possible one-time failure of the control device is reversibly intercepted by the hardware-implemented temperature monitor. If, however, an extremely unlikely major problem should occur, which extends to the hardware-implemented temperature monitor, the safety fuse will take action as a last resort for a protection. Since the action of the fuse is irreversible, no potentially endangering further use by the user may take place under these circumstances. Instead a visit to a technician or to a specialized store is to be arranged.

Preferably, the temperature of the treatment surface is already controlled by means of the control device. If the control device fails due to e.g. an error occurring in the electronics, the hardware-implemented temperature monitor allows the heating elements to be shut down independently of the control device. Even if the control device were to fail in such a way, a safety fuse would not be triggered. Only in the extremely rare case that both the control device and the hardware-implemented temperature monitor fail, for example if the corresponding components are damaged, the safety fuse guarantee a final safety control element. If an increased current demand of the heating elements occurs in the process of a strong heating up, the safety fuse cuts off the entire power supply to the device. The hierarchy of safety barriers makes it possible to intercept a one-time malfunction of the control device extremely safely. The hardware-implemented temperature monitor intervenes unnoticed and rapidly without affecting the usability of the device. An even higher safety level can be achieved by the downstream safety fuse, so that the user can be provided with an immensely effective and safe treatment device.

By serially combining the safety barriers, it can be surprisingly ensured that the treatment surface does not reach a temperature range that could endanger the patient.

In a preferred embodiment, the hardware-implemented temperature monitor comprises at least a second temperature sensor for measuring the temperature of the treatment surface and a comparator, wherein the comparator compares the temperature of the treatment surface with the maximum temperature and if the maximum temperature is exceeded, stops the current feed to the at least one heating element.

In the sense of the invention, a comparator preferentially refers to an electronic circuit for comparing two voltages, whereby the output indicates in binary form which of the two voltages is higher. In the prior art, various comparators are sufficiently well known, which are suitable for using two analog voltages to output one binary output signal and indicating which of the input voltages is higher. The Schmitt trigger may be mentioned as an example of a comparator circuit. It is preferred for a reference value for a voltage be applied to one input of the comparator using a voltage splitter. This reference value preferably corresponds to the voltage value that the second temperature sensor would show if the temperature of the treatment surface is equal to the maximum temperature. At the second input of the comparator, the output voltage of the temperature sensor, which depends on the temperature of the treatment surface, is preferably present. A particularly preferred temperature sensor has an NTC thermistor, i.e., a thermal resistor. This has a negative temperature coefficient, so that when the temperature increases, the resistance decreases and a higher current flows. However, posistors, i.e., PTC thermistors, having a positive temperature coefficient, may also be used, so that when the temperature increases, the resistance increases and a lower current flows.

If the temperature of the treatment surface rises, the voltage value at the comparator, regulated by the second temperature sensor, moves toward the voltage reference value that corresponds to the maximum temperature. As soon as the temperature exceeds the maximum temperature, the output signal on the comparator changes in a binary manner. The comparator is preferably integrated in the power supply of the heating elements. In other words, before the temperature of the treatment surface reaches the maximum temperature, the comparator preferably unblocks the supply voltage of the heating elements. However, as soon as the temperature is higher than the maximum temperature, the outlet of the comparator shuts off and interrupts the power supply to the heating elements. When the temperature of the treatment surface drops again, supply voltage is advantageously unblocked again by the comparator. As a result, reversible on and off switching of the heating elements can only take place for a time period during which the temperature of the treatment surface exceeds the maximum temperature. In addition, it may be preferred for the comparator to be unlocked by the control device when the device is turned on. Thus, if correct a start-up of the device does not take place, the comparator is configured in the setup phase to interrupt the current feed of the heating elements.

The preferred embodiment of the hardware-implemented temperature monitor described above has proven in tests to be particularly robust and reliable. Due to the reversibility of the safety switch and the simple design, the preferred embodiment is also characterized by low manufacturing and maintenance costs.

Due to the design independent of the control device and to the dedicated temperature sensor, reliable operation can be guaranteed even in the case of failure of a component of the control device. Similarly, a hardware-implemented temperature monitor in the described form using a comparator is particularly fast, since comparators are widely used electronic components that are characterized by their reliability and fast switching capability. For example, comparators with switching times of nanoseconds or less are available. Surprisingly, it was found that by using comparators in the circuit, due to their speed a particularly effective protective mechanism against overheating of the treatment surface was established.

In a preferred embodiment, the safety fuse has a threshold value for a maximum current corresponding to the heating of the treatment surface to 65° C. for 1 second. Tests have shown that only a temperature increase of more than 65° C. for more than 1 second is to be considered very critical for the sensation of pain and may lead to damage to the skin areas. It is advantageous that by adjusting the safety fuse to these parameter values, the safety fuse is not triggered prematurely in the case of an subcritical temperature increas of the treatment surface. In this manner, economic efficiency can be increased without having to make a compromise in terms of safety. Based on the electrical parameters of the heating elements, the person skilled in the art knows which safety fuse should be selected to guarantee the specified values. For this purpose, the current flow may be measured while simultaneously measuring the temperature of the treatment surface. In addition, it is particularly preferred to use a fast-acting safety fuse, which preferably reacts to a current increase within less than 20 ms. Thus it was recognized that even a short-term increase in the current for less than 20 ms can lead to a temperature elevation for more than 1 second because of the thermal inertia of the treatment surface.

Compared with non-resettable, purely temperature-dependent thermal fuses, which likewise function by melting, the current-dependent safety fuse used here has several advantages. In the case of non-resettable, purely temperature-dependent thermal fuses, the melting does not take place upon application of a current above a threshold value, but only upon application of an external temperature that is higher than a defined maximum temperature. Thus in contrast to non-resettable, purely temperature-dependent thermal fuses, current-dependent safety fuses can react even before a certain undesirable temperature is reached as a result of an elevated current acting for a relatively long period. Likewise, non-resettable, purely temperature-dependent thermal fuses always require a certain reaction time in the presence of an external temperature above a defined maximum temperature. In this way, dangerous further temperature elevations can occur. In contrast to this, current-dependent safety fuses react more quickly and with minimal system-related latency times.

In a preferred embodiment, the threshold value of the fuse is preferably between 1 A and 2.5 A, particularly preferably about 2 A. Tests have shown that with regard to the preferred heating elements, the above-mentioned threshold values guarantee with especially good reliability that the temperature of the treatment surface does not exceed a temperature of 65° C. to 70° C. for more than 1 second. By melting of the safety fuse above current values of 1 A to 2.5 A, it can thus be ensured that the temperature of the treatment surface cannot enter a range that would be hazardous to health. Thus, in the case of a normal treatment, a normal treatment current that is less than 2.5 A, preferably 1 A occurs. If a malfunction occurs, e.g., in case of continuous heating, an increased current will flow. In this case, the fuse intervenes and effectively prevents uncontrolled heating.

The advantageous selection of the maximum temperature of the hardware-implemented temperature monitor to a value between 54° C. and 58° C., preferably of about 56° C., also ensures that a large enough distance is kept to a temperature at which the fuse trips as a result of a current value above the threshold value. In this manner, accidental triggering of the safety fuse, which would result in at least a replacement of the fuse, can be avoided as long as no major malfunctioning occurs, which includes the hardware-implemented temperature monitor.

In a preferred embodiment of the invention, the device is characterized in that the device comprises a data memory for storing the system data and/or error messages. Preferred system data includes a treatment cycle counter, which preferably count the use of different types of treatment cycles separately. For example, if a shorter or a longer treatment cycle can be selected, this will be counted separately. Furthermore, the system data preferably comprises a boot counter, i.e. a counter for how often the device was started up, as well as information on the error messages with current error status.

Preferably, the following error messages can be stored: A “Reset” indicates that the voltage monitor has triggered a reset. A “Watchdog” indicates that a watchdog reset has occurred in the firmware, i.e. a system restart due to a software error. Preferably for error reporting it is possible to determine in which program mode the device was operating at the time the error occurred. A “temperature too high” may indicate that the temperature measured at the temperature sensor is too high, or that the temperature sensor is defective. A “temperature too low” may indicate that the temperature measured at the temperature sensor is too low or that the temperature sensor is defective. A “treatment temperature reached” may indicate whether the desired treatment temperature has been reached or whether an error has occurred during the preheating-up phase.

Advantageously, the stored system data and error messages can be used for diagnosis and troubleshooting for the device.

For example, these data can be read out by exchanging data with the mobile device

With the help of the data it is possible to correlate the occurred error, e.g. “temperature too high”, with further system data on the number of treatment cycles or watchdog resets. This data can thus be used to continuously optimize the safety features of the device, especially after a development phase, based on the data collected by the users. The possibility for the device to include storage of system data and error messages thus allows the continuous improvement of the hardware and software components of the device on the basis of meaningful data.

In a further preferred embodiment, the device is characterized in that a firmware is installed on the control device, which at least controls the temperature regulation of the treatment surface, wherein the firmware comprises a watchdog counter (WDC) that monitors whether the firmware is being executed. In the sense of the invention firmware is preferably understood as a software, i.e. instructions for a computer-implemented process, which is embedded in the control device, preferably a microprocessor. In other words, the firmware preferably comprises the software that is functionally linked with the hardware of the device, i.e. especially with the heating elements and temperature sensors. Preferably, the firmware is executed when the device is started and takes over the control and monitoring function of these hardware components of the device. Thus, the control device preferably evaluates the measurement data of the temperature sensors as well as user inputs on the basis of the firmware, for example, in order to control the power supply for the heating elements during the treatment cycle. In the sense of the invention, hardware-implemented components preferably designate components whose function is ensured independently of a correct execution of the firmware. As described above, the temperature monitor is hardware-implemented so that its function, i.e. limiting the maximum temperature, can be performed independently of a correct execution of the firmware on the control device. Therefore, even in the event of a system failure of the firmware, the hardware-implemented temperature monitor may rapidly and correctly limit the maximum temperature of the treatment surface.

In the particularly preferred embodiment, the firmware of the control device is monitored by means of a hardware-implemented watchdog counter. Especially preferred is a time-out watchdog. Preferably, the time-out watchdog is activated by the firmware before the start of the treatment phase. During the treatment phase, the firmware sends a signal to the time-out watchdog within a predetermined time interval to reset it. If the Time-Out-Watchdog is not reset, this will preferably lead to restarting the firmware. The time interval is preferably the time which is intended for the firmware to measure and control the temperature of the heating elements and may amount, for example, to between 2 ms and 10 ms. Such a time-out watchdog can be advantageously used to ensure that at least during the treatment phase of the device the firmware is working correctly and the temperature of the treatment surface is monitored. By means of a hardware-implemented watchdog for monitoring the firmware, preferably for example with the aid of a time-out watchdog, it can thus be ensured that if the firmware does not function correctly and the specified time interval is not observed, the treatment phase is aborted. This is a further safety feature of the device in addition to those mentioned above, which, especially in combination with the hardware-implemented temperature monitor, ensures that overheating of the treatment surface is impossible, even if the firmware is not functioning correctly,

In a preferred embodiment, the device includes at least one contact sensor which can determine whether or not the treatment surface is in contact with a skin.

In the sense of the invention, the contact sensor is a unit which, on the basis of measurement data and their analysis, can make a statement as to whether or not the treatment surface is in contact with a skin, e.g. a lip or another part of the skin. To this end preferably the contact sensor comprises a sensor or a measuring unit connected to the control device, wherein the control device may process the measurement data.

By means of the contact sensor and the information about the time at which the treatment surface contacts the skin, a particularly precise regulation of the heat flow can be realized for the treatment of itching or herpes. For example, the start of the heating-up phase can be made dependent on whether there is contact with the skin. The period of the treatment phase may also be reliably recorded in order to monitor the treatment and, if necessary, adjust further applications accordingly. The contact sensor furthermore allows improved control with regard to safety aspects. With the help of a contact sensor, it is possible to prevent the treatment surface from heating up without the knowledge or intend of the user.

In a further embodiment of the invention, the device is characterized in that the control device is able to determine whether the treatment surface is in contact with the skin on the basis of a correlation of the measurement data of a temperature sensor and data on the control of the heating element.

In another embodiment, the contact sensor is formed by a temperature sensor and a control device for controlling the heating elements. The basis of such a contact measurement is the realization that the current flow necessary to reach or maintain a temperature depends on whether the treatment surface is in contact with a thermal load (e.g. a skin). If the treatment surface is heated up when it comes into contact with the skin, heat transfer occurs, which must be compensated for by an increased energy supply to the heating elements. By evaluating the current and temperature progression, reliable statements can be made as to whether the treatment surface is in contact with the skin. Preferably to this end reference data can be provided to the control device.

In a preferred embodiment of the invention, the control device comprises reference data on a correlation of the temperature of the treatment surface with the control of the at least one heating element in the case that the treatment surface is in contact with the skin or with air. The reference data may, for example, comprise ratios of measured temperature and power supply required for this purpose. The reference data preferably include such ratios for an entire temperature curve so that by measuring the current ratio of temperature and current powe supply it can be precisely determined whether the treatment surface is in contact with the skin. It is advantageous that such a regulation can reliably distinguish not only a contact with the skin compared to air, but a also contact with the skin compared to materials with other thermal properties.

In another preferred embodiment, reference data may include the average amount of heat emitted to the skin or air. The reference data may include correlations between treatment temperature and heat output.

With such a contact sensor, a treatment temperature can be set very precisely, which corresponds to an average temperature of the external side of a treatment surface, while it is in contact with a skin.

In a further preferred embodiment of the invention, the device is characterized in that the treatment surface has a thickness between 0.2 mm and 5 mm, preferably between 0.5 mm and 2 mm, particularly preferably between 1 mm and 1.5 mm, and is made of a material having a thermal conductivity at 50° C. between 20 W/mK and 400 W/mK, preferably between 100 and 350 W/mK. The thermal conductivity (also known as heat transfer coefficient) preferably characterizes the thermal properties of the material from which the treatment surface is made. The thermal conductivity indicates the amount of heat that is conducted through the treatment surface when a temperature gradient is applied to it. In addition to thermal conductivity, the heat transport depends on the thickness of the treatment surface, the size of the treatment surface and the temperature difference between internal side of the treatment surface (contact with the heating elements) and the external side of the treatment surface (contact with the skin). Thermal conductivity is preferably expressed as the ratio of the transported heat output in watts (W) per unit temperature difference in kelvin (K) and per meter (m). However, thermal conductivity can also preferably be given as the ratio of the transported heat output in watts (W) per temperature difference in millikelvin (mK). Since the thermal conductivity can still change slightly depending on the temperature, the reference temperature is given as 50° C. The thickness of the treatment surface also refers preferably to the extent of the treatment surface between the outermost surface that contacts the skin and the innermost surface to which the heating elements are applied.

A thickness of the treatment surface between 0.2 mm and 5 mm, preferably between 0.5 mm and 2 mm and particularly preferably between 1 mm and 1.5 mm, in combination with a preferred thermal conductivity between 100 and 350 W/mK results in a therapeutically particularly effective heat transfer to the skin. In experimental tests, the preferred parameters have proven to be surprisingly advantageous. A treatment surface designed in this way avoids a too fast heat transfer to the affected skin areas which may cause an unpleasant stabbing pain sensation. Nevertheless, the heat transfer takes place in a period of time which is sufficiently abrupt to effectively activate the receptors and mask itching. Therefore, the parameters mentioned above represent an optimized selection that was not obvious for a person skilled in the art. In addition, the parameters preferably guarantee that during the treatment phase the heat of the treatment surface is rapidly and effectively transferred to the sites of the skin so that emergence of residual heat will not represent a risk.

In a preferred embodiment, the treatment surface includes ceramic or gold. It is particularly preferred for the treatment surface to be made of gold or ceramic. On the one hand, the materials ceramic and gold fall within the experimentally determined preferred ranges of thermal conductivity. Furthermore, the materials themselves do not store heat for too long, so that these materials heat and cool again relatively rapidly. This allows for increased safety, since it is possible to ensure that after the treatment phase the treatment surface will not represent a risk due to residual heat. In addition, both ceramic and gold are characterized by a high level of biological compatibility at the preferred treatment temperatures. Allergic reactions or other adverse effects can be especially effectively avoided with this choice of materials.

In a preferred embodiment, the treatment surface is directly equipped with the heating elements to ensure a better heat transfer. The person skilled in the knows suitable technologies for the direct assembly of ceramic or ceramic circuit boards, for example.

In a preferred embodiment of the invention, the device is characterized in that the treatment area is surrounded by a marker that lights up depending on the treatment cycle. For example, as a marker it may be advantageous to surround the treatment surface with a light guide. This can be illuminated, for example, during the heating phase or during the treatment phase. It has been found that the success of the hyperthermic treatment can be increased by using an explicit, glowing indication of the position of the treatment surface. For example, the visual marking promotes centered application to the affected skin parts, so that the heat pulse can be applied to these skin parts in a targeted manner. With the illuminated marker, the device can also be used in the dark, for example in a tent outdoors at night, without problems.

In another preferred embodiment, the invention relates to a kit comprising
a) a device according to the invention or a preferred embodiment thereof, and
b) a software for installation on a mobile device
   wherein the software is configured to select one of at least two treatment programs via the interface of the device.

In another preferred embodiment, the invention relates to a system comprising
a) a device according to the invention or a preferred embodiment thereof, and
b) a mobile device
   wherein a software is installed on the mobile device, which is configured to select one of at least two treatment programs via the interface of the device.

The person skilled in the art recognizes that technical features and advantages of preferred embodiments of the device according to the invention equally apply to the kit and system according the invention.

Software is preferably understood to be an application program (‘app’) that can be installed on a mobile device, such as a smartphone, in order to establish a connection to the device and perform various functions. Preferably, the interaction between the mobile device and the device is performed by means of said software. In particular, the software is configured to select one of at least two treatment programs via the interface of the device. For this purpose, the software may be configured to display the possible treatment programs on a touch display of the mobile device and to make them available for selection. Of course, the person skilled in the art is a aware of different possibilities to ensure a clear and intuitive operation. In preferred embodiments, the app can also generate a trigger or start command, so that for a preferred device mounted onto a mobile device, a treatment may be triggered directly by an actuation on the mobile device. In this case the mobile device provides the preferred operating element.

Preferred embodiments, which were disclosed in connection with the device, in particular in relation to its interaction with a mobile device, preferably equally apply for the kit and the included software. The person skilled in the art recognizes that the described interaction possibilities of a mobile device with the device can preferably be provided by the software on the mobile device. For example, a data exchange between the mobile device and the device was disclosed in a preferred embodiment and the advantages of an evaluation of the transmitted data and, if applicable, a networking with other user data were described. The person skilled in the art recognizes that it is preferred to configure an appropriate software to this end and may routinely carry out the concrete design of the software.

### DETAILED DESCRIPTION

In the following, the invention shall be explained in more detail by means of examples and figures, without intending a limitation to these.

### BRIEF DESCRIPTION OF THE FIGURES

FIG. 1 shows a preferred embodiment of the device according to the invention.

FIG. 2 shows another preferred embodiment of the device according to the invention, the device being a two-part device and comprising a head element and body element.

FIG. 3 shows a preferred embodiment of a head element of the two-part device shown in FIG. 2.

### DETAILED DESCRIPTION OF THE FIGURES

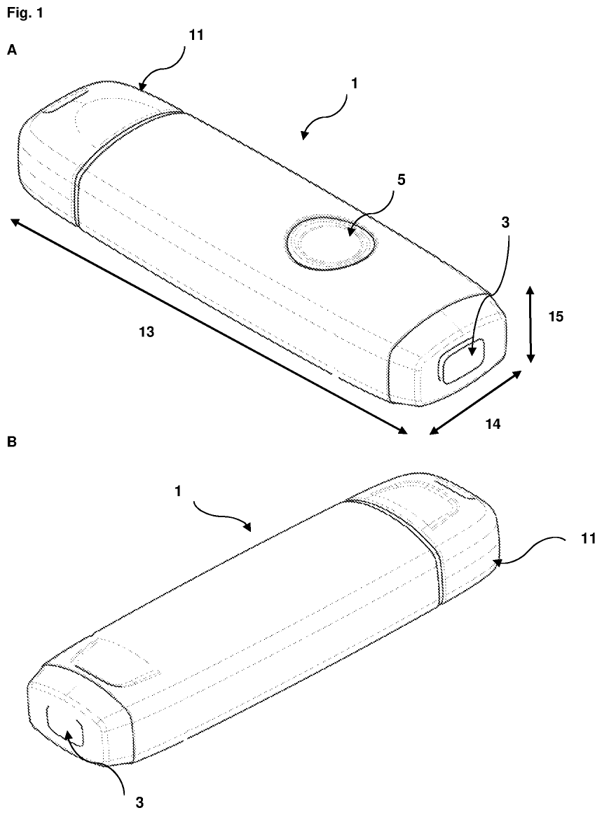

FIG. 1 shows a device for hyperthermal treatment of itching or herpes, comprising a treatment surface 3, an operating element 5 and a control device (not visible). The control device is configured to regulate the treatment surface 3 in a heating-up phase to a treatment temperature between 40°-65° C. by heating at least one heating element according to predetermined treatment parameters of a treatment program and to maintain this temperature for a treatment duration between 1 and 12 seconds. At least two different treatment programs are stored on the control device, which specify different treatment parameters. Device 1 has at least one interface (not visible) for connection to a mobile device and is configured for the selection of one of the at least two treatment programs by means of the mobile device, so that the selected treatment program is executed after actuating the operating element 5.

For communication with the mobile device, device 1 comprises a wireless interface (not visible), preferably a Bluetooth interface. Preferably, a treatment program is selected via the interface with the help of a mobile device. To this end a software (‘app’) may be provided on the mobile device. The selected treatment program is preferably executed after actuating an operating element 5 on said device 1. By actuating the operating element 5 on device 1, it is preferred that the last selected treatment program is executed. Device 1 is advantageously configured in such a way that the mobile device enables a selection of the treatment programs stored in the control device, preferably in the firmware, without the mobile device or the app being able to change operating parameters. As a result, the device 1 that can be conveniently operated by means of a mobile device may be provided without risking to compromise on safety standards.

Instead, all defined treatment programs are preferably based on well-founded empirical values or study results, wherein the possibility of a deliberately limited individualization may further improve upon patient compliance and treatment success.

Advantageously, the mobile device allows for a straightforward selection from the various treatment programs, while the device itself can be kept slim and simple. As illustrated, the device 1 preferably comprises only a single simple operating element 5 for executing the selected treatment program.

Device 1 preferably comprises a separate energy storage unit, preferably a lithium battery, for an energy supply. Device 1 may be used independently without connection to a mobile device. As a stand-alone device 1, it is particularly easy to handle and guarantees a precise positioning of the treatment surface on a site of the skin to be treated. As preferred dimensions, the device 1 may, for example, exhibit a width 14 of 15 mm to 25 mm, a thickness 15 of 10 mm to 20 mm and a length 13 of between 50 mm and 100 mm, preferably 60 mm to 80 mm.

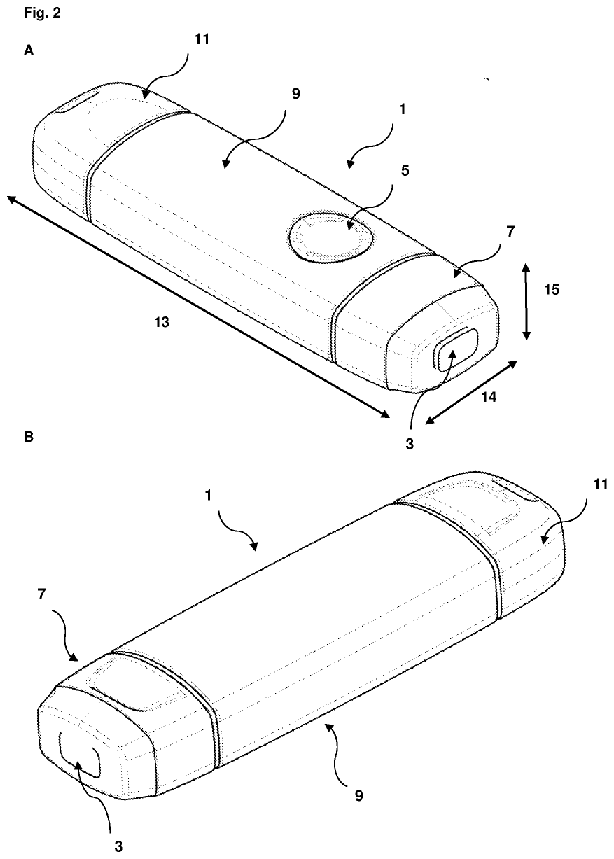

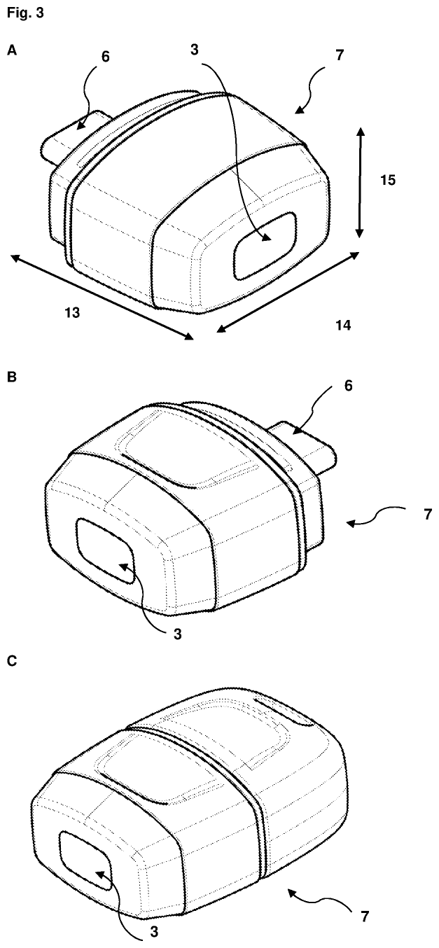

FIGS. 2 and 3 illustrate an alternative embodiment of device 1 according to the invention, wherein the device 1 is characterized by a two-part design.

The device 1 comprises a head element 7 and a body element 9, which are pluggable connected to each other via an interface 6. For the preferably illustrated embodiment, the treatment surface 3 and the control device (not visible) are present in the head element 7, while the operating element 5 and an energy storage (not visible) are present in the body element 9.

FIG. 2 shows the two-part device 1 in a connected or assembled state, while FIG. 3 shows the head element 7 in the disconnected state, allowing the interface 6 to be visible. Interface 6 may preferably allow for a USB Type C or Lightning plug connection to a mobile device. Interface 6 provides for mechanical stability in addition to other functionalities described in relation to the interface, such as data exchange and/or power supply.

As can be seen from FIGS. 2 and 3, the treatment surface 3 is located in the head element 7 on the side opposite to interface 6 (or a body element 9 connected to it). Since no energy storage is required in the head element 7, it may be kept extremely compact such that for example dimensions of a width 14 from 15 mm to 25 mm, a thickness 15 from 10 mm to 20 mm and a length 13 from 15 mm to 30 mm may be achieved.

The compact head element 7 may be mounted onto a mobile device for treatment. For this purpose, head element 7 and body element 9 are disassembled and connected to a mobile device via the available interface 6. The interface 6 now functions as an interface to the mobile device, so that in the connected or mounted state, both a selection of the treatment programs and the power supply by means of the mobile device are possible.

As in the case of the embodiment described above, a corresponding software (‘app’) may be provided on the mobile device for this purpose. However, advantageously the mobile device does not allow for making any safety-relevant changes to the treatment programs. Instead also in this embodiment, the mobile device may preferably only select the treatment programs that are stored on the control device of the head element (e.g. within the firmware). Preferably the treatment may be triggered by an appropriate operation input in an app on the mobile device.

On the other hand, device 1 may also be used advantageously as a stand-alone device, i.e. independent of a mobile device. This is preferably done in an assembled state (see FIG. 2), i.e. in a state in which head element 7 and body element 9 are connected via interface 6. Energy is preferably supplied via the energy storage in body element 9, e.g. a lithium battery.

After actuating the operating element 5, the last selected treatment program is preferably executed. Preferably said treatment program corresponds to the treatment program that was selected during a connection of head element 7 with the mobile device. However, it may also be preferred that the two-part device 1 comprises two interfaces 6 for connection with a mobile device. On the one hand, the interface 6 between a body and head element may preferably be used as a first interface to a mobile device. On the other hand, the device may comprise a further, preferably wireless interface, for example a Bluetooth interface for a second interface with the mobile device. A wireless second interface enables a connection of the device 1 with the mobile device even when the device is assembled (see FIG. 2). This means in a state in which the head element 7 is connected to the body element 9 via interface 6, so that interface 6 is not available for a connection with a mobile device.

For storage purposes, device 1 may preferably have a cap 11, which is used to protect the interface 6 on the head element 7 when disassembled or disconnected. Preferably the cap 11 as well as the body element 9 and the head element 7 are dimensioned in such a way that the cap 11 is positioned at the open end of the body element 9 in the connected state and cannot be lost.

The depicted two-part version of the device 1 allows a high degree of flexibility and ease of use, which to a large extent meet various individual requirements. As preferred dimensions, the device 1 may, for example, exhibit a width 14 of 15 mm to 25 mm, a thickness 15 of 10 mm to 20 mm and a length 13 of between 60 mm and 120 mm, preferably 70 mm to 100 mm.

In preferred embodiments, the head element 7 shown in FIG. 3 may moreover function as an independent device 1, wherein a treatment in the mounted state is carried out as described using a mobile device.

It shall be noted that different alternatives to the described embodiments of the invention may be used to carry out the invention and to arrive at the solution according to the invention. Thus, the device, system or kit according to the invention shall not be limited to the above-mentioned preferred embodiments. Rather, various embodiments are conceivable which may deviate from the solution presented. The aim of the claims is to define the scope of protection of the invention. The scope of protection of the claims is directed to cover the device, system or kit as well as equivalent embodiments of these.

### REFERENCE SIGNS

1 device

3 treatment surface

5 operating element or trigger

6 interface

7 head element

9 body element

11 cap

1-15. (canceled)

16. Device for the hyperthermal treatment of itching or herpes diseases comprising
at least one treatment surface; and
a control device, which is configured to regulate the treatment surface in a heating-up phase to a treatment temperature between 40°-65° C. by heating at least one heating element according to predetermined treatment parameters of a treatment program and to maintain said treatment temperature for a treatment duration between 1 and 12 seconds,
characterized in that at least two different treatment programs specifying different treatment parameters are stored on the control device, and wherein the device has at least one interface for a connection to a mobile device, and the device is configured for a selection of one of the at least two treatment programs by said mobile device, so that the selected treatment program is executed after actuation of an operating element which is present on the device or the mobile device,
wherein the device is configured in such a way that the mobile device is excluded from changing the treatment parameters, which are specified by the treatment programs.

17. Device according to claim 16, characterized in that the treatment parameters defined by the treatment programs include the treatment temperature and the treatment duration, wherein preferably additionally a duration for the heating-up phase is defined as a treatment parameter and/or in between 2 and 10, preferably between 4 and 8 treatment programs are stored on the control device.

18. Device according to claim 16, characterized in that the device exhibits only exactly one operating element for heating the treatment surface in accordance with the selected treatment program and a manual selection of a treatment program can be not made manually on the device itself.

19. Device according to claim 16, characterized in that the device is configured in such a way that, independently of an existing communication to a mobile device, a last selected treatment program is executed after an actuation.

20. Device according to claim 16, characterized in that the interface and the control device are configured in such a way that a selection of one of the at least two treatment programs by means of a mobile device is only allowed if an authentication has been successful.

21. Device according to claim 16, characterized in that the treatment temperatures specified in the at least two treatment programs are between 40° C. and 60° C., preferably between 45° C. and 53° C., and particularly preferably between 47° C. and 53° C.

22. Device according to claim 16, characterized in that in a first of the selectable treatment programs a treatment duration of 1-3 seconds is specified, while in a second of the selectable treatment programs a treatment duration between 3-12 seconds is specified and/or in a first of the selectable treatment programs a treatment temperature between 48° C. and 53° C. is specified, while in a second of the selectable treatment programs a treatment temperature between 45° C. and 48° C. is specified.

23. Device according to claim 16, characterized in that the size of the treatment surface is less than 1 cm², preferably between 20 and 80 mm² and/or the size of the treatment surface is between 1 cm² and 18 cm², preferably between 6 and 9 cm².

24. Device according to claim 16, characterized in that the device comprises an energy storage device, preferably a battery, particularly preferably a lithium polymer and/or a metal hydride battery, and/or the battery is a solid state battery, preferably a lithium ceramic battery.

25. Device according to claim 16, characterized in that the interface is additionally configured for a power supply by means of a mobile device and/or the interface is additionally configured for a data exchange with a mobile device.

26. Device according to claim 16, characterized in that the device comprises at least one first temperature sensor for measuring the temperature of the treatment surface and the control device sets the temperature of the treatment surface based on the measurement data of the temperature sensor, and/or a hardware-implemented temperature monitor reversibly limits the maximum temperature of the treatment surface and a fuse switches off the device in the event of a short circuit or uncontrolled continues heating.

27. Device according to claim 16, characterized in that the device comprises a contact sensor which can determine whether or not the treatment surface is in contact with a skin.

28. A kit comprising
a device according to claim 16, and
a software for installation on a mobile device,
wherein the software is configured to select one of at least two treatment programs via the interface of the device.

29. System comprising
a device according to claim 16, and
a mobile device,
wherein a software is installed on the mobile device, which is configured to select one of at least two treatment programs via the interface of the device.
